(19) United States
(12) Patent Application Publication
(10) Pub. No.: US 2016/0324989 A1
(43) Pub. Date: Nov. 10, 2016

(54) METHODS FOR ENHANCING THE DELIVERY OF ACTIVE AGENTS

(71) Applicant: WAKE FOREST UNIVERSITY HEALTH SCIENCES, Winston-Salem, NC (US)

(72) Inventors: King C. Li, Wiston-Salem, NC (US); Akiva Mintz, Greensboro, NC (US); Xiaobing Xiong, Lewisville, NC (US); Youngkyoo Jung, Clemmons, NC (US); Yao Sun, Clemmons, NC (US)

(21) Appl. No.: 15/110,211

(22) PCT Filed: Jan. 13, 2015
(86) PCT No.: PCT/US2015/011171

§ 371 Date: Jul. 7, 2016

Related U.S. Application Data
(60) Provisional application No. 61/928,526, filed on Jan. 17, 2014.

(51) Int. Cl. A61K 48/00; A61K 45/06; A61K 38/19; A61K 38/18; C07K 14/525; A61M 37/00; C07K 14/49; A61K 35/30; C07K 14/52; A61K 9/00; A61K 41/00; C12N 15/86; A61K 35/28; C07K 14/495

(52) U.S. Cl. CPC A61K 48/0083; A61K 35/28; A61K 48/0066; A61K 48/0058; A61K 45/06; A61K 38/191; A61K 38/1841; A61K 38/1866; C07K 14/525; C07K 14/495; C07K 14/49; A61K 35/30; A61K 38/19; C07K 14/52; A61K 9/0019; A61K 41/0047; C12N 15/86; A61M 37/0092; C12N 2830/002

(57) ABSTRACT

A method of increasing blood-brain barrier permeability of selected brain tissue in a subject in need thereof is carried out by: (a) parenterally administering to the subject stem cells that migrate to the brain tissue, the stem cells containing a recombinant nucleic acid, the recombinant nucleic acid comprising a nucleic acid encoding a barrier-opening protein or peptide operably associated with a heat-inducible promoter; and then (b) selectively heating the selected brain tissue sufficient to induce the expression of the barrier-opening protein or peptide in an amount effective to increase the permeability of the blood-brain barrier in the selected brain tissue. Nucleic acids, vectors, stem cells and compositions useful for carrying out such methods are also described.

### RELATED APPLICATIONS

This application claims the benefit of U.S. Provisional Patent Application Ser. No. 61/928,526, filed Jan. 17, 2014, the disclosure of which is incorporated by reference herein in its entirety.

### FIELD OF THE INVENTION

The present invention concerns methods and compositions for delivering active or therapeutic agents such as stem cells to a tissue of interest, such as neoplastic tissue in the brain.

### BACKGROUND OF THE INVENTION

Glioblastoma multiforme (GBM) is the most common and aggressive primary brain tumor, with an extremely poor prognosis (P. Wen et al., N Engl J Med. 359(5):492-507 (2008)). The dismal prognosis is a direct result of the fact that standard therapies fail to eradicate residual or infiltrating cells that reside adjacent to and infiltrate normal brain tissue. Due to their tumor-tropic migratory capacity, stem cells are emerging as feasible delivery vehicles to therapeutically target primary and invasive tumor cells. In fact, we and others have demonstrated the in vivo migratory capacity of stem cells toward primary GBM tumors as well as invasive tumor cells that intermingle with normal brain tissue (I. Germano et al., J Neurosurg. 105, 88-95 (2006); J. Dorsey et al., Mol Cancer Ther. 8, 3285-3295 (2009); A. Ashkenazi et al., J Clin Invest. 104, 155-162 (1999); D. Lawrence et al., Nat Med. 7, 383-385 (2001); H. Walczak et al., Nat Med. 5:157-163 (1999). A. Panner et al., Mol Cell Biol. 25, 8809-8823 (2005). S. Kidd et al., Stem Cells. 2009; 27(10):2614-2623 (2009)).

Two main challenges that limit stem cells as therapeutic vehicles include: (1) In addition to migrating towards tumors, stem cells are additionally attracted towards normal areas in the body that may be harmed if they non-selectively express highly toxic therapies (S. Kidd et al., Stem Cells 27(10): 2614-2623 (2009)), and (2) the low fraction of injected therapeutically engineered stem cells that migrate to the tumor limit their therapeutic potential due to the large and infeasible number of injected engineered stem cells that would be needed to induce a therapeutic response in a clinical setting. Hence there is a need for new ways to enhance the delivery of stem cell therapeutic agents, both for brain tumors such as glioblastoma multiforme and for other conditions treatable by stem cell therapy.

### SUMMARY OF THE INVENTION

While the present invention is sometimes described herein with reference to one embodiment involving the treatment of glioblastoma multiforme, those skilled in the art will appreciate that the invention may be applied to the treatment of a variety of different types of tissues, including both cancer and non-cancer tissues. Accordingly, specific discussions of glioblastoma multiforme herein are to be treated as illustrative, rather than limiting, of various aspects of the present invention.

Hence, and as discussed below, the present invention provides methods of preparing for treatment, and methods of treating, a tissue in a subject in need thereof. When considered together, the methods comprise the steps of:

(a) parenterally administering to the subject preconditioning stem cells that migrate to said tissue, said stem cells containing a recombinant nucleic acid, said recombinant nucleic acid comprising a nucleic acid encoding a stem-cell attracting chemokine operably associated with a heat-inducible promoter; then

(b) selectively heating said tissue sufficient to induce the expression of said stem-cell attracting chemokine therein in an amount effective to enhance the migration of therapeutic stem cells subsequently parenterally administered to said subject; then

(c) parenterally administering to a subject therapeutic stem cells that migrates to said tissue, said stem cells optionally (but in some embodiments preferably) containing a recombinant nucleic acid, said recombinant nucleic acid comprising a nucleic acid encoding a therapeutic agent operably associated with a heat-inducible promoter; and then optionally (but in some embodiments preferably)

(d) selectively heating said tissue sufficient to induce the expression of said therapeutic agent therein in a treatment-effective amount.

A further aspect of the invention is a method of increasing blood-brain barrier permeability of selected brain tissue in a subject in need thereof, comprising:

(a) parenterally administering to the subject stem cells that migrate to the brain tissue, said stem cells containing a recombinant nucleic acid, said recombinant nucleic acid comprising a nucleic acid encoding a barrier-opening protein or peptide operably associated with a heat-inducible promoter; and then

(b) selectively heating said selected brain tissue sufficient to induce the expression of said barrier-opening protein or peptide in an amount effective to increase the permeability of the blood-brain barrier in said selected brain tissue.

Also described herein are pharmaceutical formulations containing stem cells as described above, and further below, in a pharmaceutically acceptable carrier, for use in carrying out the methods described herein.

Also described herein are stem cells for use in preparing a pharmaceutical formulation as described herein, and for use in the methods as described herein.

The present invention is explained in greater detail in the drawings herein and the specification set forth below. The disclosures of all US Patent references cited herein are to be incorporated herein by reference in their entirety.

### BRIEF DESCRIPTION OF THE DRAWINGS

FIG. 1A. In vitro migratory potential of neural stem cells (NSCs) in response to chemo-attractants secreted by tumor cells. Representative fluorescent microscopy (FM) and light microscopy (LM) photomicrographs of filters show migrated NSCs, which indicate that the cells migrated from the TRANS WELL to the plate. The migration was quantitated by taking photographs under fluorescent microscopy and counting cells that had migrated from the TRANS WELL to the plate surface (arrows). A histogram comparing the migration in the presence of GBM-conditioned media versus control (CTRL, serum-free media) indicate a significantly increased migration in the presence of the GBM-conditioned media. Data are expressed as mean±SEM (n=3); *, P=0.0432, Students' t-test.

FIG. 1B. GFP-expressing NSCs migrate toward glioblastoma in vivo. Images of various sections demonstrated that GFP-expressing NSCs (arrows) colocalized to primary tumors (dashed lines) and infiltrative projections (expressing DsRed), but not in normal brain, indicating the in vivo GBM-tropism of NSCs.

FIG. 2A. Schematic of lentivirus vector, pLenti-pHSP70:FLuc/GFP-pRSV:RFP, containing HSP70 promoter (P_{HSP70}) driving expression of reporter genes, green fluorescent protein (GFP) and firefly luciferase (F-Luc), which are separated by an internal ribosome entry sites (IRES) for proportional expression. In addition to HSP70 driven expression, the plasmid also constitutively expresses red fluorescent protein (RFP) via the RSV promoter (P_{RSV}) to visualize and select for cells successfully transduced.

FIG. 2B. HSP70-driven reporter gene expression after heating at various temperatures. Jurkat cells were transduced with pLenti-pHSP70:FLuc/GFP-pRSV:RFP and heated in a PCR thermal cycler for 30 minutes at the temperatures indicated. The cells were replated in a 96-well plate and cultured for 18 hours under normal culture conditions. Cells were exposed to luciferin and imaged using an IVIS 100 imaging system. Light signal was quantitated by drawing regions of interest (ROI) around the wells and plotting the light intensity in the histogram.

FIG. 3A. Demonstration of dual reporter expression of HSP70-driven F-Luc/GFP and constitutive RFP (Const.) in Jurkat cells. Virus was infected into Jurkat cells and once transduction was confirmed by RFP expression, cells were heated to 43° C. in a PCR thermal cycler for the indicated period of time. Following heating, cells were replated and cultured under normal culture conditions for 24 hours. After exposure to luciferin, bioluminescence and multicolor fluorescence (GFP and RFP) images were recorded (RFP, upper left; phase constrast, upper right; GFP, lower left; merged, lower right). Light signal was quantitated and plotted in the histogram.

FIG. 3B. Demonstration of dual reporter expression of HSP70-driven F-Luc/GFP and constitutive RFP in B16F10 melanoma cells. Virus was infected into melanoma cells and once transduction was confirmed by RFP expression, cells were heated to 43° C. in a PCR thermal cycler for the amounts of time indicated. Following heating, cells were replated and cultured under normal culture conditions for 24 hours. After exposure to luciferin, bioluminescence and multicolor fluorescence (GFP and RFP) images were recorded (RFP, upper left; phase constrast, upper right; GFP, lower left; merged, lower right). Light signal was quantitated and plotted in the histogram.

FIG. 4. HSP70-driven reporter gene expression in NSCs. NSCs were infected with pLenti-pHSP70:FLuc/GFP-pRSV:RFP virus and 24 hours after transduction, plasmid expression was confirmed via the constitutive expression of RFP. NSCs were then heated to 43° C. in a PCR thermal cycler for 30 minutes, replated and cultured under normal cell culture conditions for 24 hours. Multicolor fluorescence imaging demonstrated that heating induced the HSP70 promoter, resulting in GFP expression. RFP, upper left; phase constrast, upper right; GFP, lower left; merged, lower right.

FIG. 5. In vivo HSP70-driven firefly luciferase expression in implanted cells after high intensity focused ultrasound (HIFU)-controlled heating. B16F10 melanoma cells (8×10⁵) that stably expressed the pLenti-pHSP70:FLuc/GFP-pRSV:RFP vector were subcutaneously implanted into the right lower thigh of C57BL/6 mice. Seven days after cell implantation, the injection site was heated to 43° C. for 30 minutes with magnetic resonance thermometry (MRT)-guided HIFU. Eight hours after HIFU-induction, mice were imaged for bioluminescence after being anesthetized and injected i.p. with 150 mg/kg D-luciferin (Xenogen). Bioluminescence was detected with the IVIS 100 In Vivo Imaging System (PerkinElmer) (upper panel). Regions of interest (ROI) were drawn over the B16 injection sites and photon flux was quantitated and graphed for all ROI (lower panel).

FIG. 6A. In vivo MRT-guided HIFU. Continuous ultrasound exposure was performed to heat the brain tissue in a rat cadaver at a selected focal spot (arrow). Temperature was monitored in real-time using MRT; ambient temperature: 37° C., TE/TR: 8.7/30 ms, 5 slices, 10 cm FOV, 128×128 matrix, 0.78×0.78 mm² in-plane resolution, 5 mm slice thickness and 18 seconds temporal resolution,

FIG. 6B. MRT-guided HIFU feedback loop. HIFU sequences were applied (upper panel). Real-time MRT was used to monitor exact temperature (dashed line) and thermal dose (solid line) changes within the target spot during a 15 minute HIFU experiment (lower panel). The thermal dose in the graph represents cumulative equivalent minutes at 43° C.

FIG. 7A. Real-time MRT. The tumor location was identified (arrow) with T2-weighted images (TE/TR: 5.7/17 ms, 88 slices, 10 cm FOV, 256×256 matrix and 0.4 mm isotropic spatial resolution) and the intracranial temperature was monitored in real-time using MRT (TE/TR: 8.7/30 ms, 5 slices, 10 cm FOV, 128×128 matrix, 0.78×0.78 mm² in-plane resolution, 5 mm slice thickness, and 18 sec. temporal resolution). Temperature changes within the tumor core (9 voxels) are shown during the HIFU experiment (right panel).

FIG. 7B. pHSP70-driven reporter expression after HIFU. Eight and 48 hours after HIFU-induction, rats were imaged for bioluminescence after being anesthetized and injected i.p. with 150 mg/kg D-luciferin (Xenogen). Signal demonstrated robust F-Luc expression (circle) in the rat treated with HIFU in contrast to the control that had the same number of tumor cells with reporter construct, but not heated with HIFU.

FIG. 7C. Quantitation of bioluminescence signal. Regions of interest were drawn over the brain sites and photon flux was quantitated and graphed at 8 and 48 hours after HIFU treatment.

FIG. 8. Schematic of the targeted HIFU-activated therapeutic drug delivery strategy. Step 1: Recombinant, dormant stem cells are injected intravenously. Step 2: Stem cells directionally migrate toward primary tumor (T), invasive projections, and microsatellite tumors. Step 3: HIFU waves non-invasively heat the tumor and surrounding tissue to 43° C. under constant monitoring by MRT. Step 4: HIFU-induced heating activates stem cell expression of potent therapeutic via the heat shock promoter. NB, normal brain; TZ, infiltrating tumor zone; HZ, HIFU-induced therapeutic zone.

FIG. 9. Schematic of therapeutic construct used in conjunction with image-guided HIFU. The lentivirus vector contains the HSP70 promoter (P_{HSP70}) driving expression of sTRAIL and F-Luc, which are separated by an internal ribosome entry site (IRES) for proportional expression. In addition, the vector also constitutively expresses red fluorescent protein to visualize and select for cells that have been successfully transduced.

FIG. 10A. GFP-expressing NSCs, transfected with a vector encoding sTRAIL and mCherry transgenes under control of the CMV promoter, demonstrate expression of the sTRAIL, GFP and mCherry transgenes with no accompanying NSC death or rounded cells detected at 72 hours post-transfection.

FIG. 1013. The media from sTRAIL transfected NSC cultures kills GBM cells. NSCs, transfected with a vector encoding sTRAIL and mCherry transgenes under control of the CMV promoter, were grown in culture media, the media was transferred to separate wells containing GBM cells, and cell death was monitored. This analysis indicated that even very low amounts of sTRAIL present in unconcentrated media could kill GBM cells. In contrast, control media (CTRL) from NSCs mock transfected with blank vector expressing only mCherry did not have any effect on GBM cells. Data are expressed as mean±SEM (n=16). ***, P=0.0008, Student's t-test.

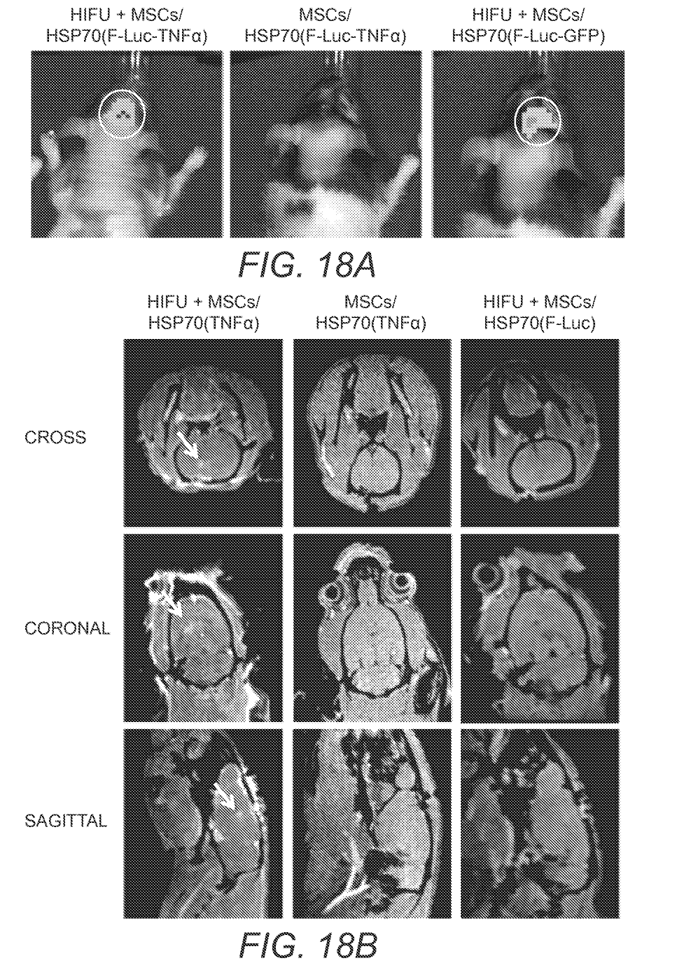

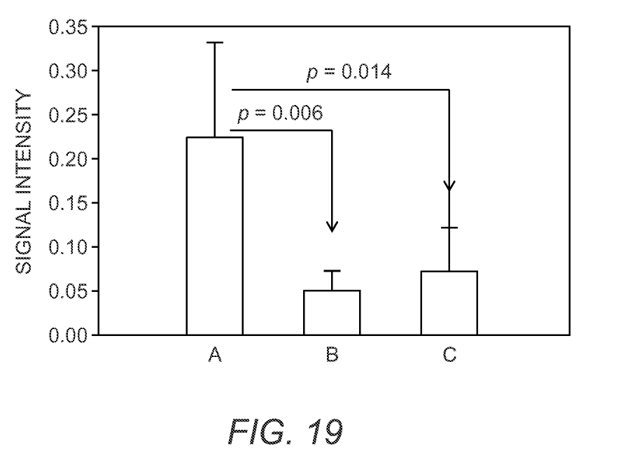

FIG. 10C. NSCs that stably express sTRAIL kill GBM cells. U251MG GBM cells (1×10³) that constitutively express F-Luc were co-incubated with NSCs (1×10³) that stably express sTRAIL. After 48 hours, GBM cell viability was measured using bioluminescence. Results indicated a complete obliteration of GBM cells exposed to sTRAIL-secreting cells compared to GBM cells that were co-incubated with control cells that did not express sTRAIL.

FIG. 11. Magnetic resonance image of intracranial human GBM tumor in rat brain.

FIG. 12A. Schematic of lentivirus vector, pLenti-HSP70 (F-Luc-2A-Cytokines), containing HSP70 promoter driving expression of firefly luciferase and cytokines, Tumor Necrosis Factor α (TNFα), Transforming Growth Factor β1 (TGFβ1) or Vascular Endothelial Growth Factor (VEGF), which are separated by an internal ribosome entry sites for proportional expression. In addition to HSP70-driven expression, the plasmid also constitutively expresses red fluorescent protein (RFP) and blasticidin S deaminase (BSD) to visualize and select for cells successfully transduced. 5′LTR, 5′ long terminal repeat; Ψ, packaging signal; RRE, Rev response element; cPPT, central polypurine tract; WPRE, Woodchuck hepatitis virus Post-transcriptional Regulatory Element; 3′LTR (SIN), 3′ long terminal repeat with SIN mutation.

FIG. 12B. Human mesenchymal stem cells (upper panels) and NSCs cells (lower panels) tranduced with lentiviral vectors expressing GFP, cytokines and RFP.

FIG. 13. Stem cell migration analysis. Stem cells, transduced with pLenti-HSP70 (F-Luc-2A-cytokines and suspended in serum-free DMEM, were mildly heated to 43° C. by a heat block, ultrasound or infrared light for 20 minutes and seeded in the wells of the lower compartment of a TRANS WELL plate. Stem cells expressing GFP in serum-free DMEM were seeded in the upper compartment. The TRANS WELL system was incubated in a CO₂ incubator and the number of cells that migrated into the lower compartment was counted under a fluorescence microscope. Stem cells without heat induction were seeded in the lower compartment as the control.

FIG. 14. In vitro migration of hMSCs in response to cytokines secreted by hMSCs induced with mild heating by heat block (HB), ultrasound (US) and infrared light (IR). Total number of cells per mm² are indicated.

FIG. 15. In vitro migration of NSCs in response to cytokines secreted by NSCs induced with mild heating by heat block (HB), ultrasound (US) and infrared light (IR). Total number of cells per mm² are indicated.

FIG. 16A. Presence of TNFα in media from SF767 human glial tumor cells transduced with a lentiviral vector encoding TNFα under control of the HSP70 promoter. Cells heated a first time (1^{st}) exhibited a high level of TNFα. Cells boosted by an additional heating period (2^{nd}), which was 24 hours after the initial heating, exhibited an even higher level of TNFα expression. TNFα in media was measured 16 hours after each heating by an ELISA assay. Non-induced cells were used as the control (CTRL).

FIG. 16B. Migration of NSCs in response to conditioned media from SF767 cells transformed to express TNFα under the control of the HSP70 promoter. RFP-expressing NSCs were incubated with SF767 cells in a TRANSWELL plate (8 μM pores) for 24 hours to assess the migratory response of NSCs to TNFα Migration was quantified by taking photographs under fluorescent microscopy and counting cells that had migrated from the TRANSWELL to the plate surface. The assay was performed in triplicate. Representative photomicrographs of filters (upper panels) showed that NSCs (indicated by arrows) migrated from the TRANWELL to the plate containing SF767 cells. Data demonstrate that conditioned media from heat-induced SF767 cells induced NSC migration compared to media from control (CTRL) unheated cells (lower panel).

FIG. 17A. Schematic of lentiviral plasmid, containing HSP70 promoter driving expression of reporter gene (luciferase), cytokines (TNFα), and RSV promoter driving expression of RFP and selectable marker blasticidin gene.

FIG. 17B. Transduced MSCs (red) with pLenti-HSP70 (TNFα-Luc)-RSV (RFP-BSD).

FIG. 17C. Heat-activated luciferase and TNFα expression.

FIG. 18A. F-Luc expression activated by MRI-guided HIFU in combination with MSCs implanted in the brain in a rat. 18 hours after HIFU induction, rats were imaged for bioluminescence after being anesthesized and injected i.p. with 150 mg/kg d-luciferin.

FIG. 18B. BBB opening activated by MRI-guided HIFU in combination with MSCs implanted in the brain in a rat. Contrast enhanced T1 images were acquired 2 days after HIFU induction. Contrast agent is administered by i.v. injection.

FIG. 19. Quantification analysis of BBB opening activated by MRI-guided HIFU in combination with MSCs implanted in the brain in a rat. A) Rats (N=6) with MSCs-HSP70 (Luc-2A-TNFα) and HIFU treatment, B) rats (N=4) implanted with MSCs-HSP70 (Luc-2A-TNN without HIFU treatment, and C) rats (N=4) implanted with MSCs-HSP70 (Luc-2A-GFP) with HIFU treatment.

### DETAILED DESCRIPTION OF ILLUSTRATIVE EMBODIMENTS

The present invention is primarily concerned with the treatment of human subjects, but the invention may also be carried out on animal subjects, particularly mammalian subjects such as dogs, cats, livestock and horses for veterinary purposes. While subjects may be of any suitable age, the subjects are in some embodiments neonatal, infant, juvenile, adolescent, adult, or geriatric subjects.

“Treat” as used herein refers to any type of treatment that imparts a benefit to a patient, particularly delaying or retarding the progression disease, or relieving a symptom of that disease.

“Pharmaceutically acceptable” as used herein means that the compound or composition is suitable for administration to a subject to achieve the treatments described herein, without unduly deleterious side effects in light of the severity of the disease and necessity of the treatment.

“Concurrently” as used herein means sufficiently close in time to produce a combined effect (that is, concurrently may be simultaneously, or it may be two or more events occurring within a short time period before or after each other).

“Nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. The term nucleic acid is used interchangeably with gene, cDNA, and MRNA encoded by a gene.

““Heterologous nucleic acid” generally denotes a nucleic acid that has been isolated, cloned and ligated to a nucleic acid with which it is not combined in nature, and/or introduced into and/or expressed in a cell or cellular environment other than the cell or cellular environment in which said nucleic acid or protein may typically be found in nature. The term encompasses both nucleic acids originally obtained from a different organism or cell type than the cell type in which it is expressed, and also nucleic acids that are obtained from the same cell line as the cell line in which it is expressed.

“Nucleic acid encoding” refers to a nucleic acid which contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences which may be introduced to conform with codon preference in a specific host cell.

“Recombinant” when used with reference to a nucleic acid generally denotes that the composition or primary sequence of said nucleic acid or protein has been altered from the naturally occurring sequence using experimental manipulations well known to those skilled in the art. It may also denote that a nucleic acid or protein has been isolated and cloned into a vector, or a nucleic acid that has been introduced into or expressed in a cell or cellular environment other than the cell or cellular environment in which said nucleic acid or protein may be found in nature.

“Recombinant” or when used with reference to a cell indicates that the cell replicates or expresses a nucleic acid, or produces a peptide or protein encoded by a nucleic acid, whose origin is exogenous to the cell. Recombinant cells can express nucleic acids that are not found within the native (nonrecombinant) form of the cell. Recombinant cells can also express nucleic acids found in the native form of the cell wherein the nucleic acids are re-introduced into the cell by artificial means. Such a cell is “transformed” by an exogenous nucleic acid when such exogenous nucleic acid has been introduced inside the cell membrane. Exogenous DNA may or may not be integrated (covalently linked) into chromosomal DNA making up the genome of the cell. The exogenous DNA may be maintained on an episomal element, such as a plasmid. In eucaryotic cells, a stably transformed cell is generally one in which the exogenous DNA has become integrated into the chromosome so that it is inherited by daughter cells through chromosome replication, or one which includes stably maintained extrachromosomal plasmids. This stability is demonstrated by the ability of the eucaryotic cell to establish cell lines or clones comprised of a population of daughter cells containing the exogenous DNA.

### Heat Inducible Promoters.

Any suitable heat inducible promoter may be used to carry out the present invention, examples of which include but are not limited to HSP70 promoters, HSP90 promoters, HSP60 promoters, HSP27 promoters, HSP25 promoters, ubiquitin promoters, growth arrest or DNA Damage gene promoters, etc. See, e.g., U.S. Pat. Nos. 7,186,698; 7,183,262; and 7,285,542; See also I. Bouhon et al., Cytotechnology 33: 131-137 (2000) (gad 153 promoter).

### Pro-Migratory Cytokines.

A variety of pro-migratory cytokines (which may also be referred to as “stem cell-attracting chemokines,” and the nucleic acids encoding them, are known and can be used to carry out the present invention. Examples include, but are not limited to, TNF-alpha, stromal cell-derived factor 1 alpha (SDF-1 alpha), tumor-associated growth factors, transforming growth factor alpha, fibroblast growth factor, endothelial cell-derived chemoattractants, vascular endothelial growth factor (VEGF), stem cell factor (SCF), granulocyte colony-stimulating factor (G-CSF), and integrins. See, e.g., U.S. Pat. No. 8,569,471 (all of which may be mammalian, such as human).

### Blood-Brain Barrier Opening Agents.

A variety of agents are known to open the blood-brain barrier in a manner beneficial to enhancing the delivery of therapeutic or diagnostic agents administered into the blood to brain tissue. See, e.g., Examples of such agents include, but are not limited to opening protein or peptide selected from the group consisting of bradykinin, thrombin, endothelin-1, substance P, platelet activating factor, cytokines (e.g., IL-1alpha, IL-1beta, IL-2, IL-6, TNFalpha), macrophage inflammatory proteins (e.g., MIP-1, MIP-2), and complement-derived polypeptide C3a-desArg.

### Therapeutic Agents.

A variety of different therapeutic agents (generally protein or peptide therapeutic agents) and the nucleic acids encoding them, are known that can be used to carry out the present invention. In general, such agents are toxins, fragments of toxins, drug metabolizing enzymes, inducers of apoptosis, etc. Particular examples include, but are not limited to, bacterial toxins, plant toxins, fungal toxins and combinations thereof; kinases; and inducers of apoptosis such as PUMA; BAX; BAK; BcI-XS; BAD; BIM; BIK; BID; HRK; Ad E1B; an ICE-CED3 protease; TNF-related apoptosis-inducing ligand (TRAIL); SARP-2; and apoptin (including active fragments thereof). See generally US Patent Application Publication No. 20130310446; see also U.S. Pat. Nos. 8,450,460; 7,972,812; 7,736,637; and 5,763,233.

### Recombinant Nucleic Acids and Vectors.

Techniques for the production of recombinant nucleic acids, in which a promoter as described above is operatively associated with a nucleic acid encoding a pro-migratory cytokine, blood brain barrier opening agent, or therapeutic agent as described above, are known. Examples include but are not limited to those described in U.S. Pat. No. 7,186,698 to Moonen and U.S. Pat. No. 7,183,262 to Li et al.

Vectors into which such recombinant nucleic acids can be inserted, ligated, or otherwise associated, and useful for carrying out the invention are likewise known. Examples include but are not limited to DNA viral vectors, RNA viral vectors, plasmids, ballistic particles, etc.

### Stem Cells and Transformed Stem Cells.

Stem cells may be stably or transiently transformed with a recombinant nucleic acid by any suitable means, with or without the use of a vector as described above. Suitable stem cells and methods and vectors for their transformation, propagation, formulation and administration are known. Examples include but are not limited to those set forth in U.S. Pat. Nos. 6,368,636; 6,387,367; 7,022,321; 8,034,329; 8,057,789; 8,216,566; and 8,518,390. The stem cells may be collected from any suitable tissue or biological fluid, such as placenta, amniotic fluid, blood, umbilical cord blood, etc. In general, the stem cells may be embryonic, adult, or induced pluripotent stem cells, with the specific choice of stem cell depending upon the specific condition and/or tissue for which they are intended.

### Pharmaceutical Formulations, Dosage and Administration.

Stem cells for use in carrying out the present invention (including but not limited to those described above) may be formulated for administration in a pharmaceutically acceptable carrier in accordance with known techniques. See, e.g., Remington, The Science And Practice of Pharmacy (9^{th} Ed. 1995). Formulations of the present invention suitable for parenteral administration comprise sterile aqueous and non-aqueous injection solutions of the active compound(s), which preparations are preferably isotonic with the blood of the intended recipient. These preparations may optionally contain anti-oxidants, buffers, bacteriostats and solutes which render the formulation isotonic with the blood of the intended recipient. Aqueous and non-aqueous sterile suspensions may include suspending agents and thickening agents.

Parenteral administration of the stem-cell containing pharmaceutical formulations may be through any suitable route, including but not limited to intraveneous, intrarterial, subcutaneous, intramuscular, and intraperitoneal injection. The number of stem cells delivered in any particular administration will depend upon a variety of factors, such as the type of stem cell being administered, the age, weight, and condition of the subject, the tissue and condition being treated, etc., but in general will be from one, five, or ten million cells, up to one, five, ten or fifty billion cells, or more.

### Tissues for Treatment.

A broad variety of different tissues, including neoplastic and non-neoplastic, are known targets for stem cell treatment. See, e.g., V. Segers and R. Lee, Stem-cell therapy for cardiac disease Nature 451, 937-942 (2008); S. Kim and J. de Vellis, Stem cell-based cell therapy in neurological diseases: A review, J Neurosci. Res. 87, 2183 (2009); A. Caplan, Review: Mesenchymal Stem Cells: Cell-Based Reconstructive Therapy, in Orthopedics, Tissue Engineering, 11, 1198-1211 (2005), etc.

Hence, as noted above, in some embodiments, the tissue for treatment is a neoplastic or cancer tissue, examples of which include but are not limited to brain cancer tissue or tumors (e.g. gliomas such as glioblastoma multiforme, meningiomas, pituitary adenomas, nerve sheath tumors, etc.), breast cancer tissue or tumors, skin cancer tissue or tumors (e.g., melanoma, basal cell skin cancer, squamous cell skin cancer, etc.) prostate cancer tissue or tumors, lung cancer tissue or tumors, ovarian cancer tissue or tumors, colon and colorectal cancer tissue or tumors, pancreatic cancer tissue or tumors, etc.

In other embodiments, the tissue for treatment is non-neoplastic or non-cancerous tissue, but injured or diseased tissue suitable for stem cell treatment. Examples of such tissue include but are not limited to central nerve, peripheral nerve, retina, skeletal muscle, cardiac muscle, epidermal, liver, pancreatic, skeletal, endocrine, and exocrine tissue, (e.g., where the aforesaid tissue is afflicted with an acute injury, anoxic injury, metabolic disease, or autoimmune disease). Particular examples include, but are not limited to, treating acute or chronic brain injury, acute spinal-cord injury, heart damage, hematopoiesis, baldness, missing teeth, deafness, blindness and vision impairment, motor neuron diseases, graft vs. host disease, Crohn's disease, neural and behavioral birth defects, diabetes, etc.).

### Heating of Selected Tissue.

The particular manner of heating the selected tissue (sufficient to induce expression of the gene operatively associated with the heat-inducible promoter) will depend upon the particular tissue or target tissue being heated. In general, the selectively heating step may carried out by ultrasound, laser, radiofrequency, microwave or water bath. See, e.g., U.S. Pat. No. 7,186,698 to Moonen and U.S. Pat. No. 7,183,262 to Li et al. Thus for deep tissue (e.g., located in brain or other internal organ) the localized or selected heating may be carried out invasively or non-invasively. Suitable alternatives include, but are not limited to, a catheter with a heat tip, a catheter with an optical guide through which light or laser light beam can be directed (e.g., an infrared light) and by focused ultrasound (which can be delivered by any of a variety of different types of apparatus; see, e.g., U.S. Pat. Nos. 5,928,169; 5,938,608; 6,315,741; 6,685,639; 7,377,900; 7,510,536; 7,520,856; 8,343,050). The extent to which the selected tissue is heated will depend upon factors such as the choice of particular promoter, the duration of heating, and the tissue chosen for heating, but in general may be up to about 1 or 2 degrees centigrade to 5 or 6 degrees centigrade, for 1, 5, 10, or 15 minutes, or more.

### Enhancing Blood-Brain Barrier Permeability.

Enhancing blood-brain barrier permeability is an ongoing goal (see, e.g., U.S. Pat. No. 8,349,822), and the materials and methods described herein may be used or adapted to methods of enhancing blood-brain barrier permeability, Such a method of increasing blood-brain barrier permeability of selected brain tissue in a subject in need thereof, generally comprising: (a) parenterally administering to the subject stem cells that migrate to the brain tissue, said stem cells containing a recombinant nucleic acid, said recombinant nucleic acid comprising a nucleic acid encoding a barrier-opening protein or peptide operably associated with a heat-inducible promoter; and then (b) selectively heating said selected brain tissue sufficient to induce the expression of said barrier-opening protein or peptide in an amount effective to increase the permeability of the blood-brain barrier in said selected brain tissue (e.g., so that concurrent or subsequent delivery of an active therapeutic or diagnostic agent to the selected tissue is enhanced, including but not limited to preconditioning or therapeutic stem cells as described herein, or other active agents such as therapeutic antibodies and chemotherapetic agents).

For example, the stem cells (and the selective heating) can be administered in an amount effective to increase the cytotoxic effect of a therapeutic agent drug in said subject, said method further comprising administering the therapeutic agent to the subject Any suitable therapeutic agent for which enhanced BBB permeability would be advantageous may be used, examples of which include but are not limited to therapeutic stem cells (including but not limited to those described above), protein and peptide therapeutic or diagnostic agents (e.g., diagnostic and therapeutic monoclonal antibodies (including active binding fragments thereof)), or chemotherapeutic drugs. Specific examples include but are not limited to temozolomide (“Tmz”), VP-16, paclitaxel, carboplatin, tumor necrosis factor-related apoptosis-inducing ligand (“TRAIL”), troglitazone (“TGZ”), pioglitazone (“PGZ”), rosiglitazone (“RGZ”), and ciglitazone (“CGZ”), procarbazine, vincristine, BCNU, CCNU, thalidomide, irinotecan, isotretinoin, imatinib, etoposide, cisplatin, daunorubicin, doxorubicin, methotrexate, mercaptopurine, fluorouracil, hydroxyurea, vinblastine, and combinations thereof. Composition, dosage and administration of the stem cells, and heating, may be as described above, and composition, dosage and administration of the other therapeutic or diagnostic active agent may be carried out in accordance with known techniques for specific agents, or variations thereof that will be apparent to those skilled in the art. See, e.g., U.S. Pat. No. 8,450,460; see also U.S. Pat. Nos. 8,628,778; 8,580,258; 8,449,882; 8,445,216; 8,409,573; 5,624,659; and 5,558,852.

The present invention is explained in greater detail in the following non-limiting Examples.

### EXAMPLES

### Example 1

### Materials and Methods

Transduction of Jurkat and B16F10 Melanoma Cells.

Viral particles were custom generated by Gentarget, Inc (San Diego, Calif.). Twenty microliters of particles (1×10⁷ IFU/ml) (mixed with polybrene at a 1:1 ratio) were contacted with cells in a 24-well plate and centrifuged (1200 RPM at 32° C.) for 60 minutes. Cells were subsequently incubated overnight under normal cell culture conditions (37° C./15% CO₂). Successful viral transduction was confirmed by expression of RFP, which is driven by the constitutive RSV promoter. Following confirmation of viral infection, cells were precisely heated to the appropriate temperature (37° C.-45° C.) using a PCR thermal cycler (T-gradient, Biometra) for an appropriate length of time (e.g., 5-50 minutes). Bioluminescent and GFP signal resulting from the pHSP70 driven F-Luc/GFP were measured by standard methods. See, e.g., J. Dorsey et al., Mol. Cancer Ther. 8(12):3285-3295 (2009); S. Wang et al., Cancer Biol. Ther. 6(10):1649-53 (2007).

Recombinant Stem Cell.

A lentiviral expression plasmid, pLenti-Hsp70 (F-Luc-2A-cytokine)-RSV (RFP-BSD), which contains (a) the heat-inducible HSP70 promoter driving expression of firefly luciferase (F-Luc) and different cytokines that can attract stem cell migration, and (b) the RSV promoter driving expression of red fluorescent protein (RFP) and optionally blasticidin selection marker (BSD), was constructed. The attracting cytokines used in this study included tumor necrosis factor alpha (TNFα), vascular endothelial growth factor (VEGF) and transforming growth factor beta 1 (TGFβ1). The RFP reporter and BSD, under control of the regular RSV promoter, were used to sort and/or select transduced cells for long-term expression via flow cytometry or blasticidin (BSD) antibiotics. Human mesenchymal stem cells (hMSCs) or rat neural stem cells (rNSC) were transduced with this construct using a lentiviral vector (GenTarget, San Diego, Calif.). Briefly, stem cells were seeded in 24-well plates at 1×10⁴ cells per well and grown overnight. The medium was replaced with fresh warm complete medium (0.5 ml), followed by addition of an appropriate amount of lentivirus solution to obtain the desired multiplicity of infection (MOI). Cells were then centrifuged at 800×g for 1 hour at 34° C., and then maintained at 37° C. in a humidified atmosphere containing 5% CO₂ for another 72 hours. Cell fluorescence was checked under a fluorescence microscope. Further, the transduced cells were screened by addition of an appropriate amount of blasticidin. NSCs or hMSCs were also transduced with pLenti(GFP) to express green fluorescence protein.

Stem Cell Migration.

In vitro cell migration analysis was performed to compare the effects of different cytokines on the migration of stem cells with a 13D FALCON FLUOROBLOK TRANSWELL chamber system. hMSC transduced with pLenti-Hsp70 (F-Luc-2A-cytokine)-RSV (RFP-BSD) were grown to 60-80% confluence in T-75 flasks. The cells were trypsinized, suspended in serum-free DMEM, and divided into two aliquots. One aliquot of the stem cells was mildly heated to 43° C. by a heat block (HB), ultrasound (US) or infrared light lamp (IR) for 20 minutes, while another aliquot was incubated at 37° C. as the control. A total of 20,000 heat-induced hMSCs diluted in 700 μl serum-free DMEM were seeded in the wells of the lower compartment of a 24-well chamber, and hMSCs (˜5×10³) expressing GFP were seeded into the upper compartment in triplicate. Stem cells without heat induction and suspended in serum-free DMEM were seeded in the wells of the lower compartment as the negative control. The TRANSWELL system was incubated in a CO₂ incubator and after 48 hours of incubation, the cells that had migrated into the lower compartment were counted under a fluorescence microscope.

### Example 2

### Tumor Tropism of Neural Stem Cells

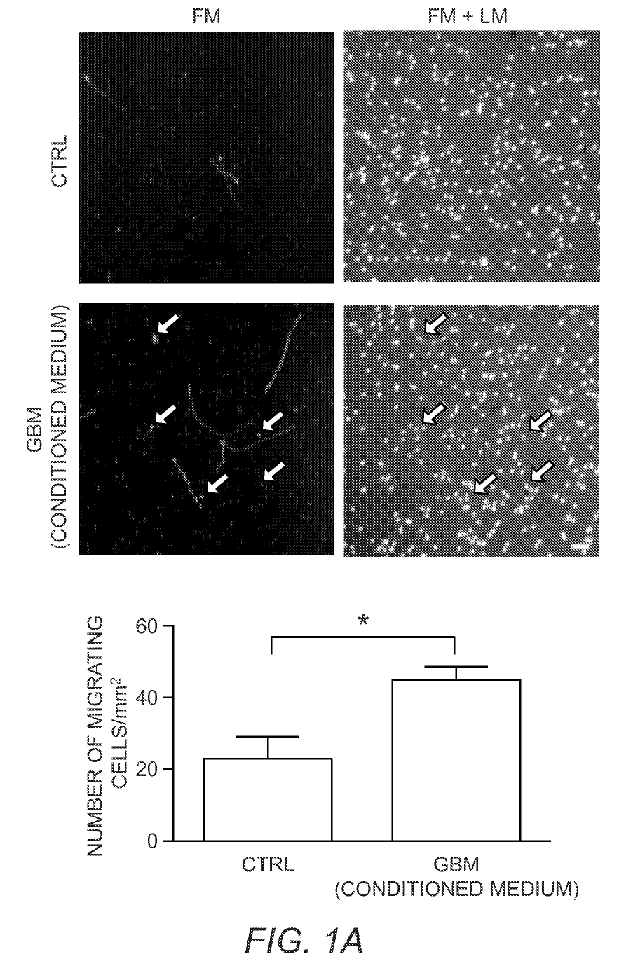

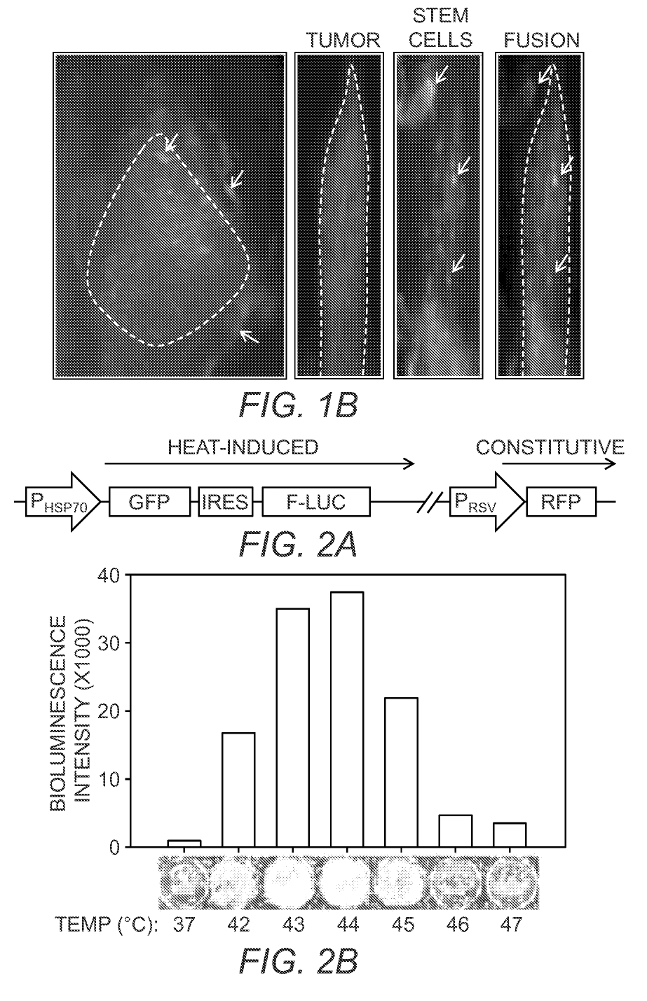

The migratory ability of GFP-expressing neural stem cells (NSCs, (Stemcell Technologies Inc, Vancouver, Canada) in response to conditioned medium from a GBM cell line (for 24 hours) was determined using a TRANS WELL plate (8 μm pores). This analysis indicated that NSCs exhibit GBM tropism in vitro (FIG. 1A). To determine whether this response also occurred in vivo, athymic nude mice were injected with human GBM tumor cells expressing DsRed. Seven days post-tumor implantation, 5×10⁵ GFP-expressing NSCs were implanted 2 mm from the tumor. Animals were sacrificed at day 15 post-tumor injection and the brains were fixed in PFA (4%) and analyzed. This analysis indicated that NSCs also exhibit GBM tropism in vivo (FIG. 1B). The results of these analyses are consistent with previous results demonstrating that stem cells, including mesenchymal stem cells (MSCs) and NSCs exhibit GBM tropism in vivo (I. Germano et al., J. Neurosurg. 105(1):88-95 (2006); J. Dorsey et al., Mol. Cancer Ther. 8(12):3285-3295 (2009); A. Ashkenazi et al., J Clin. Invest. 104(2):155-162 (1999); D. Lawrence et al., Nat. Med. 7(4):383-385 (2001); H. Walczak, et al., Nat. Med. 5(2):157-163 (1999); A. Panner et al., Mol. Cell Biol. 25(20):8809-8823 (2005); K. Aboody et al., Proc. Natl. Acad. Sci. USA 97(23):12846-12851 (2000); S. Benedetti et al., Nat. Med. 6(4):447-450 (2000); F. Davis et al., J. Neurosurg. 88(1):1-10 (1998); L. Sasportas et al., Proc. Natl. Acad. Sci. USA 106(12):4822-4827 (2009); M. Ehtesham et al., Expert Rev. Neurother. 3(6):883-895 (2003); P. Kabos et al., Expert Opin. Biol. Ther. 3(5):759-770 (2003); M. Ehtesham et al., Cancer Res. 62(24):7170-7174 (2002); A. Birbrair et al., PLoS One. 6(2):e16816 (2011); A. Birbrair et al., Stem Cell Res. 10(1):67-84 (2013); A. Birbrair et al., Exp. Cell Res. 319(1):45-63).

### Example 3

### Controlled Expression of pHSP70 In Vitro

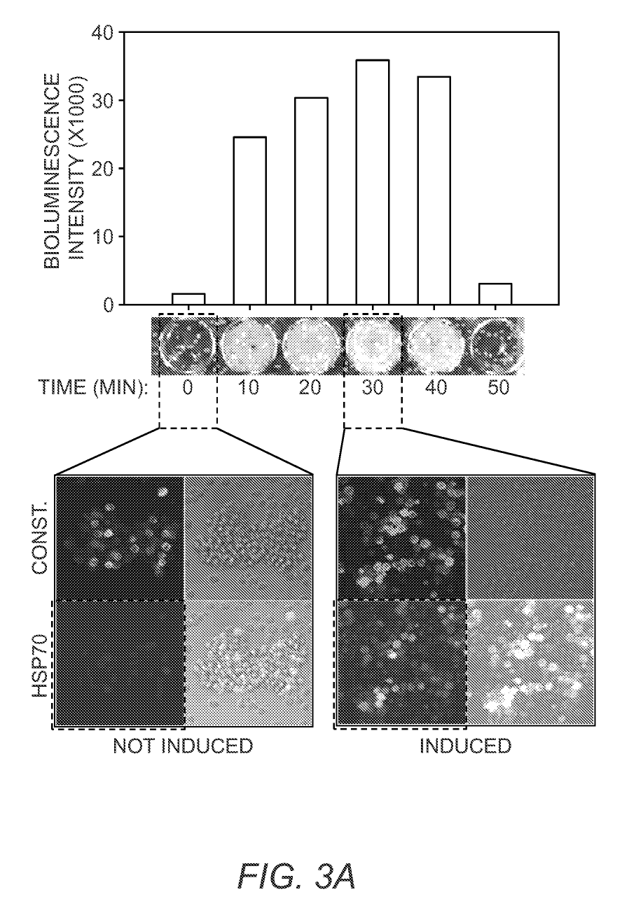

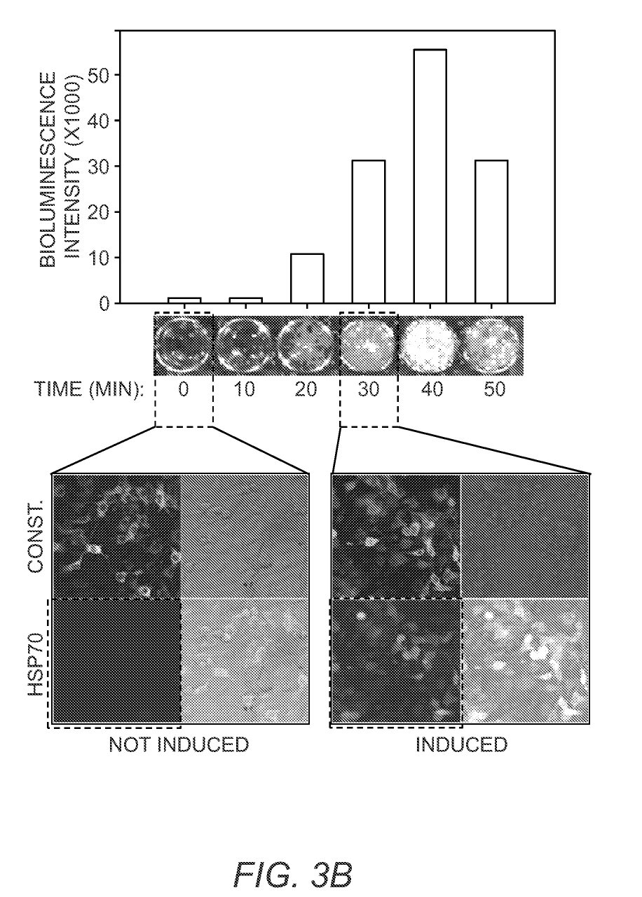

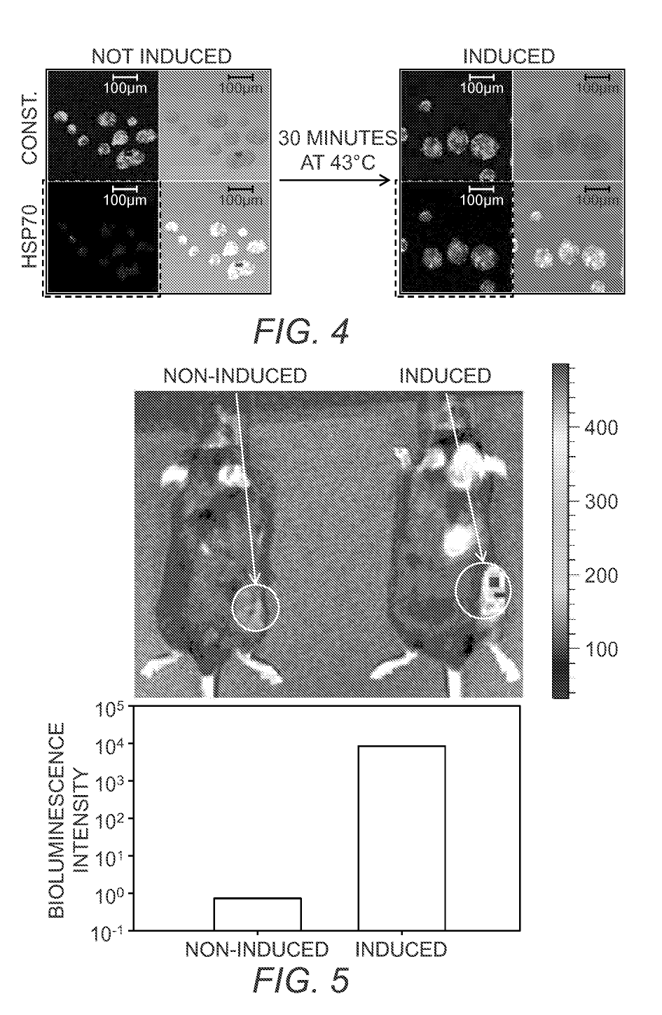

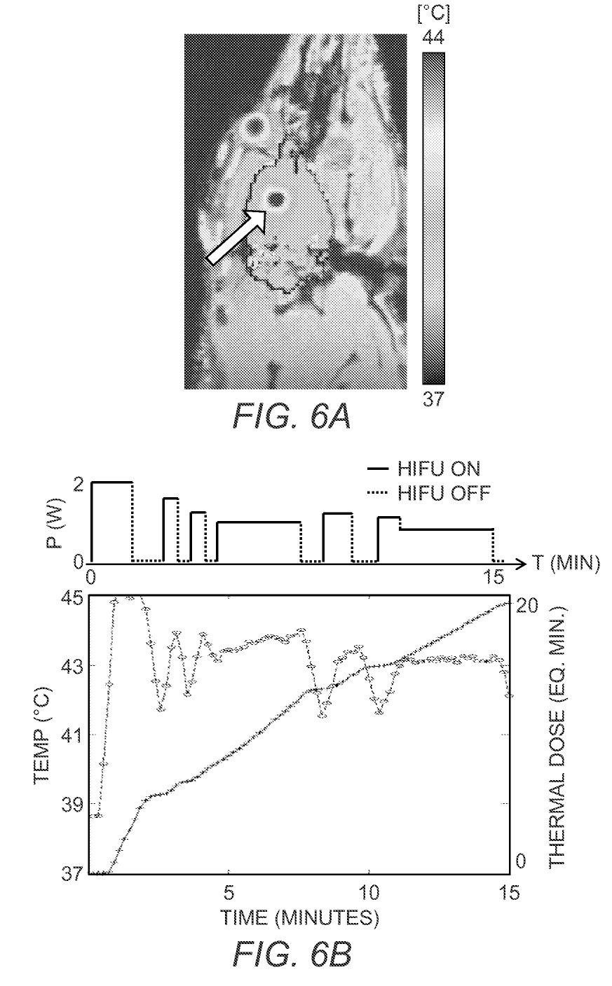

HSP70 expression is highly regulated and can be induced via non-toxic mild heating (G. Li & J. Mak, Cancer Res. 45(8):3816-3824 (1985); J. Landry et al., Cancer Res. 42(6):2457-2461 (1982); J. Landry et al., Int. J. Radiat. Oncol. Biol. Phys. 8(1):59-62 (1982); J. Subjeck & T. Shyy, Am. J. Physiol. 250(1 Pt 1):C1-17 (1986); S. Flanagan et al., Am. J. Physiol. 268(1 Pt 2):R28-32 (1995); K. Kregel et al., J. Appl. Physiol. 79(5):1673-1678 (1995); K. Diller, Annu. Rev. Biomed. Eng. 8:403-424 (2006)). It was therefore posited that HSP70 could be used to noninvasively and artificially modulate therapeutic gene expression in vivo in a spatial and temporal controlled manner. Thus, a viral construct was prepared, which was designed to concurrently express pHSP70-controlled firefly luciferase (F-Luc) and green fluorescent protein (GFP) reporter genes, in combination with constitutively expressed red fluorescent protein (RFP) reporter for confirmation of construct integration (FIG. 2A). Using this vector, designated pLenti-pHSP70:FLuc/GFP-pRSV:RFP, viral particles were custom generated by Gentarget, Inc (San Diego, Calif.) and infected into Jurkat cells. Cells were heated in a PCR thermal cycler (T gradient, Biometra) for 30 minutes at various temperatures and replated into a 96-well plate for 18 hours under normal cell culture conditions. Cells were subsequently exposed to luciferin and reporter protein expression was analyzed. The results demonstrated that HSP70-driven expression of luciferase was tightly dependent on temperature and peaked at 43-44° C. in Jurkat cells (FIG. 2B). To determine the timing of HSP70-driven gene expression, pLenti-pHSP70:FLuc/GFP-pRSV:RFP was transduced into Jurkat (FIG. 3A) and B16F10 melanoma (FIG. 3B) cells and heated at 43° C. for varying lengths of time. The results of this analysis indicated that an increase in bioluminescent signal was obtained as heat exposure time increased. Signal peaked at 30-40 minutes and decreased thereafter, likely due to decreasing viability as the length of heating time exceeded 50 minutes.

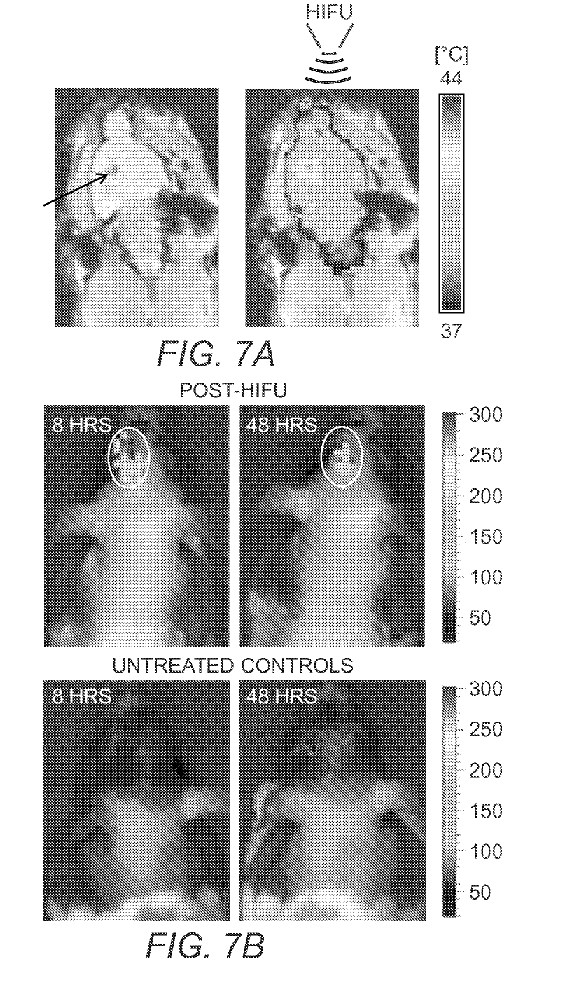

To demonstrate the use of the pLenti-pHSP70:FLuc/GFP-pRSV:RFP in stem cells, NSCs were tranduced with the viral vector and pHSP70-driven expression of luciferase in response to mild heating (43° C. for 30 minutes) was measured (FIG. 4). This analysis confirmed that the dual promoter design could be used in stem cells and is therefore of use as a tumor-tropic therapeutic vehicle.

### Example 4

### In Vivo HSP70-Driven Firefly Luciferase Expression in Implanted Cells

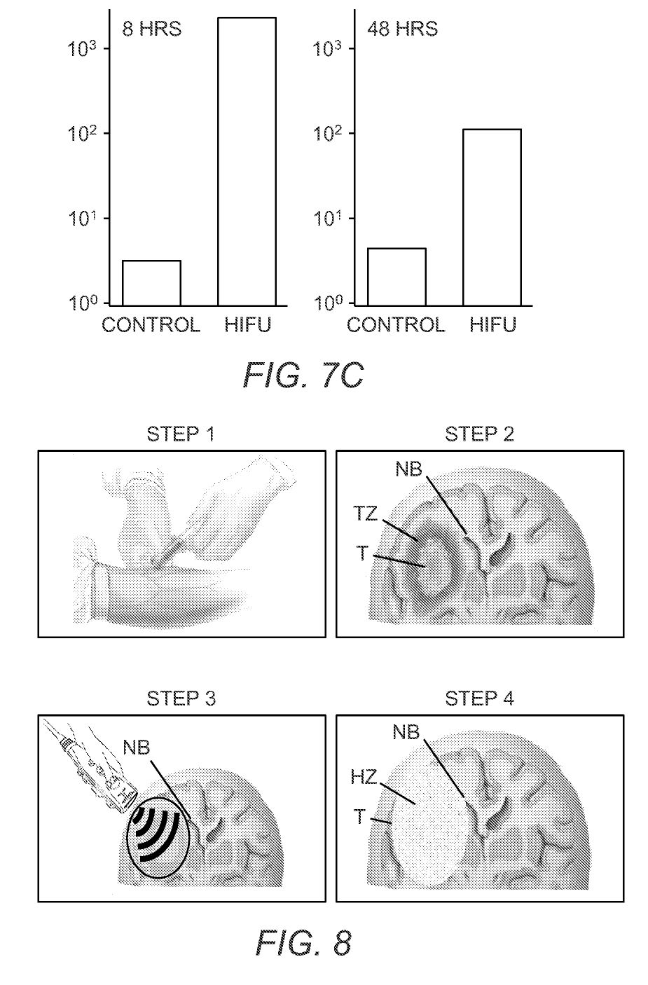

High intensity focused ultrasound (HIFU) is a non-invasive translational way of mildly heating tumor and/or surrounding normal tissue to non-toxic temperatures (˜43° C.). Using a number of model systems, the ability of HIFU to precisely heat tissue to non-toxic temperatures, including normal brain tissue has been demonstrated (B. O'Neill et al., J. Magn. Reson. Imaging 35(5):1169-1178 (2012); B. O'Neill et al., Ultrasound Med. Biol. 35(3):416-424 (2009); K. Hynynen et al., J. Acoust. Soc. Am. 132(3):1927 (2012); K. Hynynen & J. Sun, IEEE Trans. Ultrason. Ferroelectr. Freq. Control. 46(3):752-755 (1999)). Therefore, it was determined whether magnetic resonance thermometry (MRT)-guided HIFU could be used safely in vivo to non-invasively heat recombinant stem cells and induce pHSP70-driven expression of transduced genes. For this analysis, B16 melanoma cells were stably infected with pLenti-pHSP70:FLuc/GFP-pRSV:RFP and subcutaneously implanted in mice. Tumor tissue was gently heated using HIFU and bioluminescent signals were measured. This analysis indicated that a very specific and strong induction of bioluminescent signal was observed 8 hours after HIFU induction (FIG. 5). In contrast, unheated tumors did not exhibit significant luciferase signal, indicating the specificity of this approach.

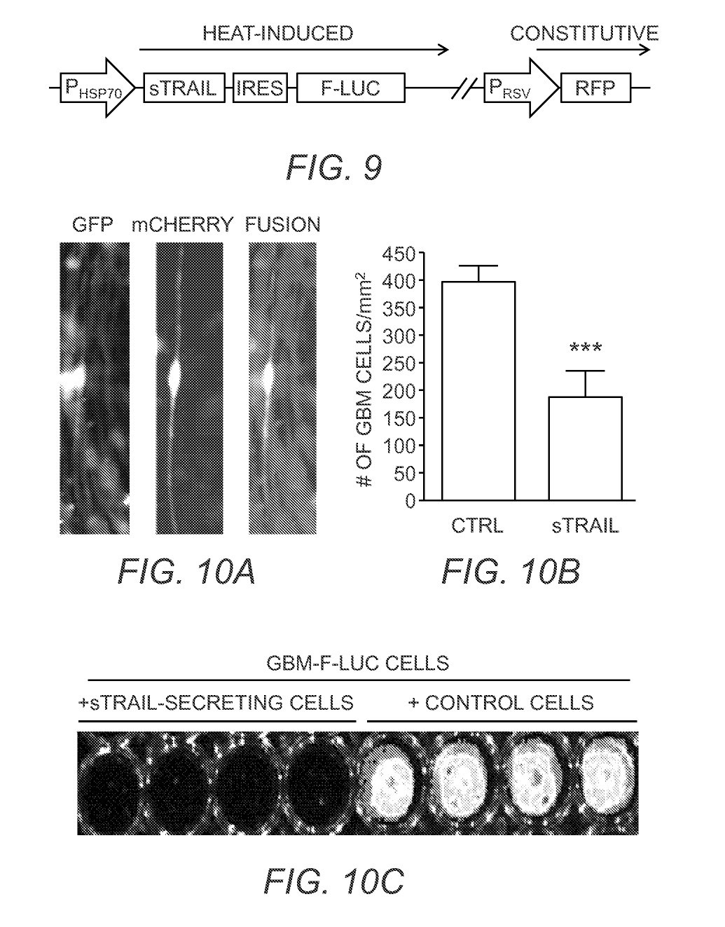

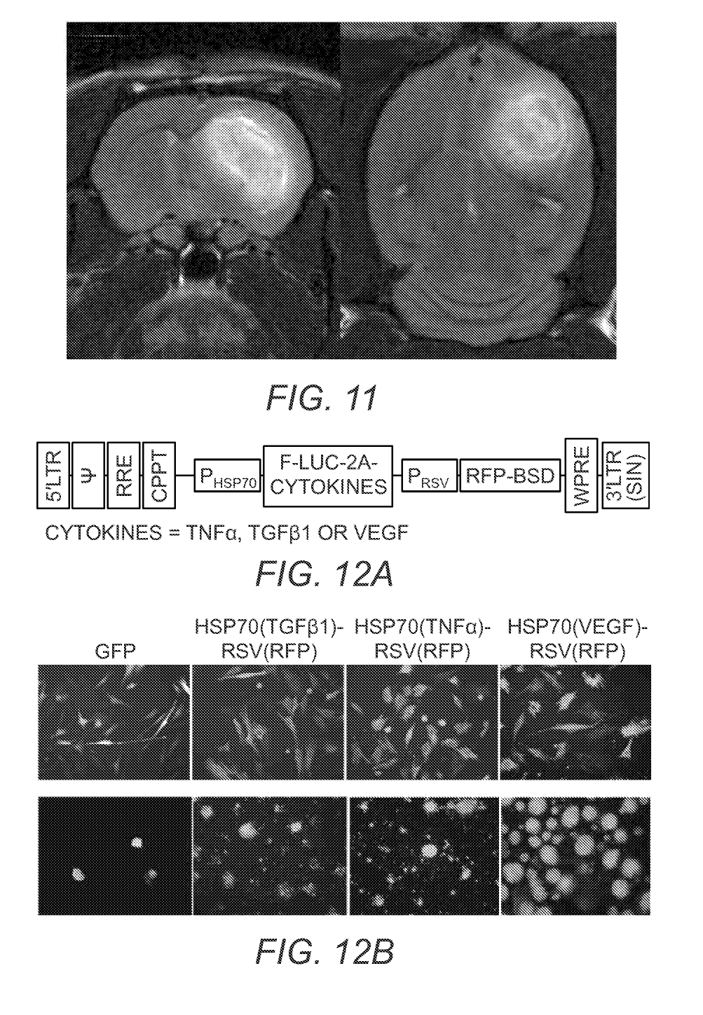

In addition to subcutaneous experiments, the transduced B16 cells were implanted intracranially into rat brains and, 7 days after implantation, HIFU with the guidance of MR thermometry was used to heat the implanted cells. Analysis of the rat cadaver indicated that the MR thermometry-guided HIFU treatment protocol successfully heated the intracranial cells and induced expression of the F-Luc reporter, as shown by bioluminescent signal (FIGS. 6A and 6B). These data demonstrate that MRT can serve as a feedback for adjustment of the HIFU and maintain a near-constant target temperature.

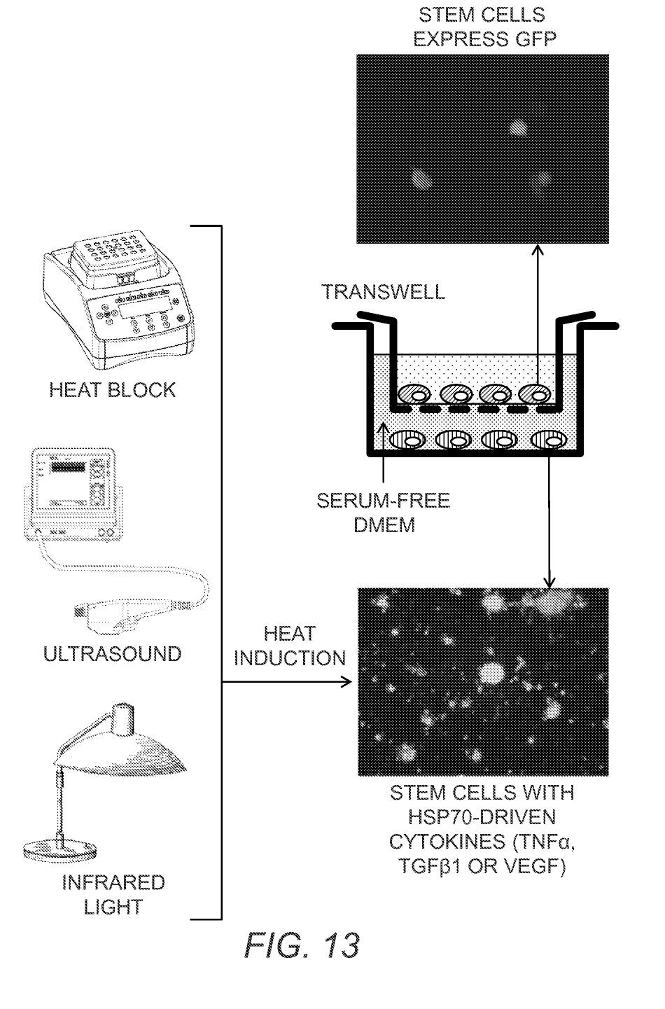

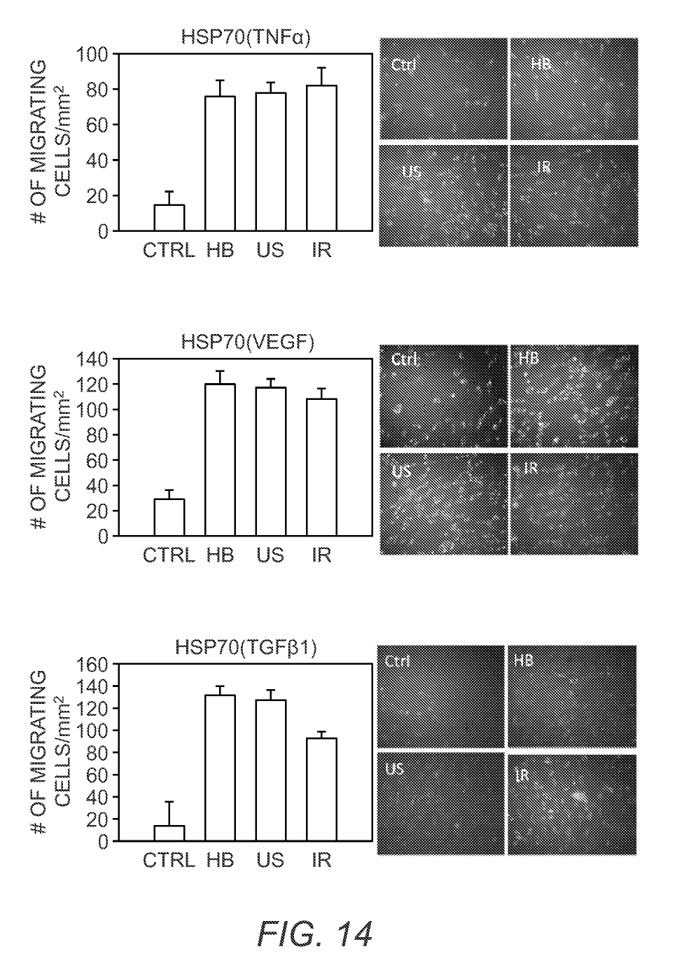

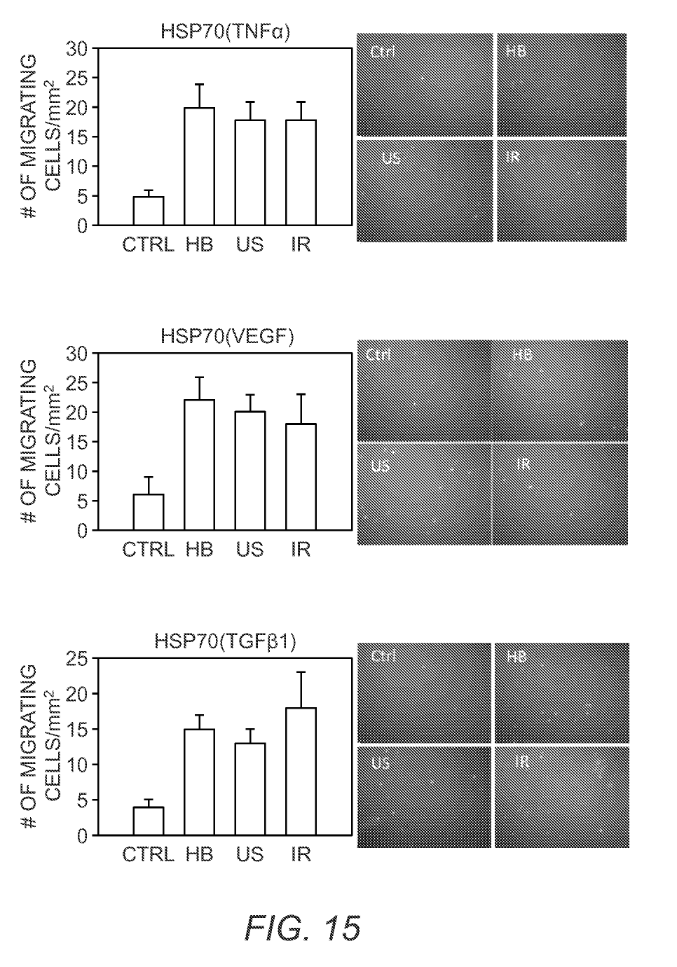

Having established that MRT-guided HIFU can be used in viva, HIFU-induced HSP70 driven luciferase gene expression was analyzed in implanted B16F10 melanoma cells that stably express the reporter construct pLenti-pHSP70:FLuc/GFP-pRSV:RFP. Transduced B16F10 cells (5×10⁵ cells) were stereotactically implanted into the right frontal lobe of athymic nude rats, and 7 days after implantation, the tumor was heated to 43° C. for 30 minutes with MRI-guided HIFU. Eight and 48 hours after HIFU-induction, rats were imaged for bioluminescence (FIGS. 7A and 7B). Quantitation of bioluminescence (FIG. 7C) indicated the feasibility and specificity of inducing reporter genes using image-guided HIFU in an intracranial setting.

### Example 5

### HSP70-Driven sTRAIL Expression and In Vivo Efficacy

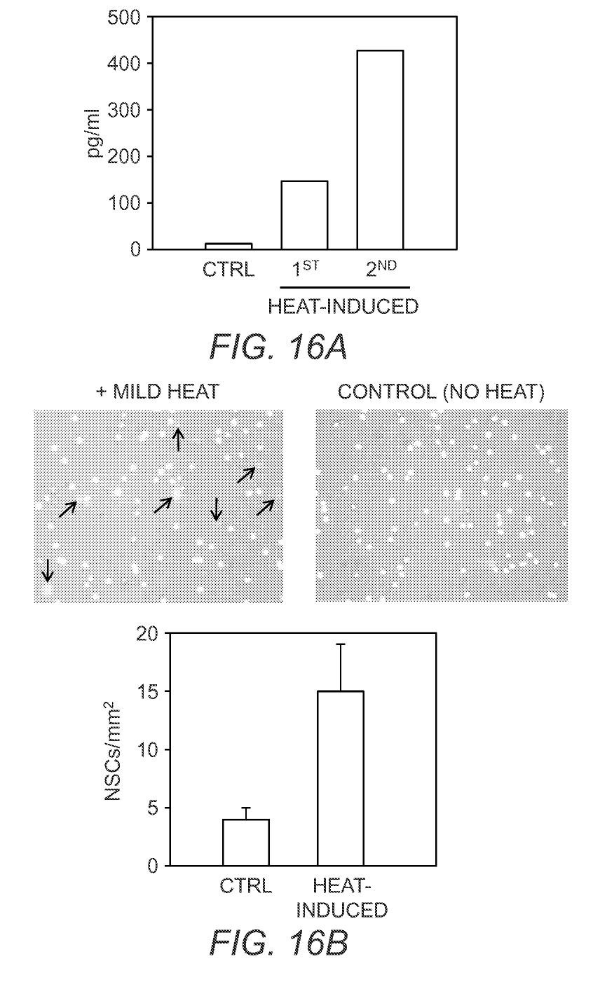

GBM is an invariably fatal malignancy due to its aggressive nature as well as the poor accessibility it offers potential therapeutics. Systemically administered therapeutics typically have limited ability to significantly penetrate the blood brain barrier (BBB), resulting in high likelihood of systemic toxicity before reaching therapeutic levels in the central nervous system (CNS). Other approaches, such as local delivery, often suffer from inconsistent delivery and high local toxicities due to the high concentrations reached uniformly in normal brain tissue adjacent to the tumor (S. Kunwar et al., Neuro. Oncol. 12(8):871-881 (2010); J. Sampson et al., J. Neurosurg. 113(2):301-309 (2010)). In contrast, tumor-tropic cell-based therapies can deliver high concentrations of therapeutics to the tumor microenvironment due to their tendency to aggregate in the primary tumor or adjacent to infiltrative tumor cells (S. Kidd et al., Stem Cells 27(10):2614-2623 (2009); A. Nakamizo et al., Cancer Res. 65(8):3307-3318 (2005)). However, cell-based strategies that rely on constitutive expression of therapeutics have the potential to expose non-tumor tissue to potentially toxic therapeutics. This is especially true when therapeutics are delivered systemically, as it has been demonstrated that a large number of cells migrate through normal tissue (S. Kidd et al., Stem Cells 27(10):2614-2623 (2009)). By comparison, the present invention encompasses the use of image-guided HIFU to activate recombinant stem cells to express potent anti-cancer therapeutics (e.g., via the HSP70 promoter) only in the tumor and peritumoral area that are temporally targeted via image guidance. See FIG. 8. Advantageously, technology that delivers HIFU through the human skull to a depth of the operator's choosing has been used in clinics for other applications (R. Medel et al. Neurosurgery 71(4):755-763 (2012)).

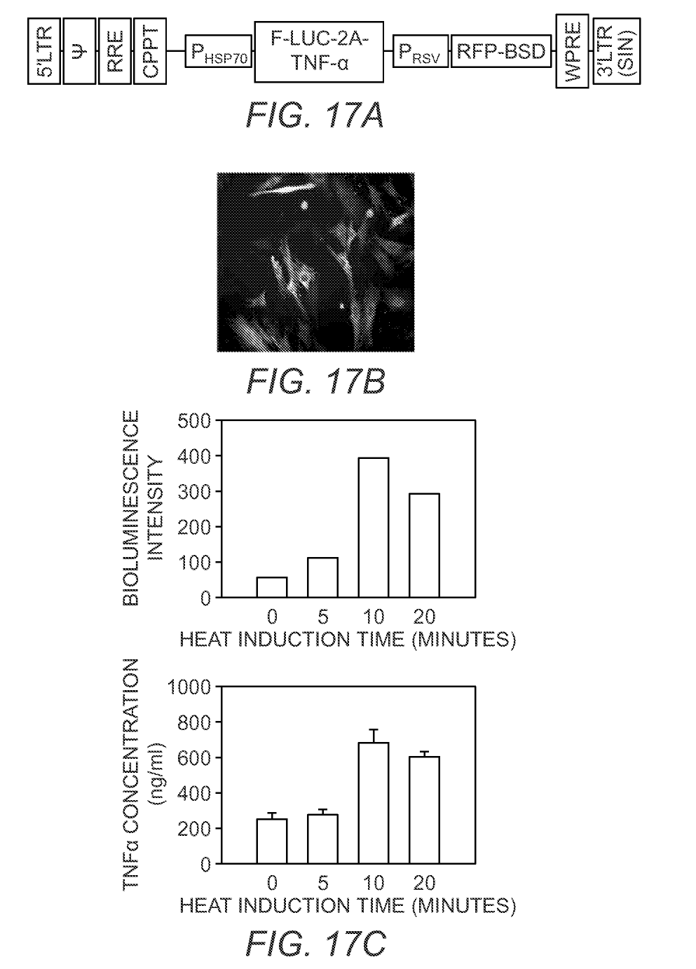

To demonstrate the use of the HIFU remote activation platform to deliver a therapeutic agent, soluble TRAIL (sTRAIL) is used as a prototype therapeutic for the treatment of GBM. The open reading frame encoding sTRAIL is inserted into the lentiviral construct to generate pLenti-pHSP70:sTRAIL/FLuc-pRSV:RFP (FIG. 9), which concurrently expresses (a) sTRAIL under the control of the HSP70 promoter, and (b) F-Luc as an imaging reporter. In addition, the construct constitutively expresses RFP to allow for the enrichment of sTRAIL expressing cells using fluorescence activated cell sorting (FACS). Using this construct, various types of stem cells (e.g., MSCs and NSCs) are infected with the therapeutic viral construct. Cells are induced at different temperatures to maximize pHSP70-controlled expression and cell viability. To evaluate therapeutic induction in stem cells (at 8 and 24 hours post heating), the expression of secreted sTRAIL is measured via western blot analysis, as well as bioluminescent signal resulting from induced F-Luc expression. In addition, the in vitro anti-tumor activity of sTRAIL-containing media obtained from stem cells transduced with pLenti-pHSP70:sTRAIL/FLuc-pRSV:RFP is determined using a standard colorimetric MTS/PMS assay (Promega) (A. Mintz et al., J. Neurooncol. 64(1-2):117-123 (2003); A. Mintz et al., Neoplasia 4(5):388-399 (2002); V. Nguyen et al., Translational Oncology 4(6):390-400 (2011); V. Nguyen et al., Neuro-Oncology 14:1239). It is expected that heated stem cells expressing sTRAIL will kill GBM cells. Indeed, it was found that NSCs could be transduced to express and secrete sTRAIL into the media and kill GBM cells (FIGS. 10A-10C). To confirm that sTRAIL expression does not alter tumor-tropic migration toward GBM, migration of transduced NSCs and MSCs in conditioned media is tested with established GBM cell lines (e.g., U251, U87, G48a) using the TRANSWELL method described herein.

For in vivo analysis, rats bearing invasive orthotopic GBM tumors are used (G. Kitange et al., J. Neurooncol. 92(1):23-31 (2009); J. Sarkaria et al., Mol. Cancer Ther. 6(3):1167-1174 (2007); J. Sarkaria et al., Clin. Cancer Res. 12(7 Pt 1):2264-2271 (2006)). This clinically relevant model involves direct engraftment of patient tumor specimens into the flank of nude mice or rats. These tumors are removed and be expanded/maintained by subsequent serial passage in the rodent flank. By way of illustration, 1×10⁶ human GBM cells in 10 pit PBS were implanted intracranially in athymic rats. Stereotactic injection was accomplished with a 10 μL syringe (Hamilton Co., Reno, Nev.) with a 30-gauge needle, inserted 3.5 mm deep through the burr hole, mounted on a digital stereotactic apparatus (David Kopf Instruments, Tujunga, Calif.). A 5 mm burr hole was created with a surgical drill (Harvard Apparatus, Holliston, Mass.) 1.5-2 mm left of the midline and 1-1.5 mm posterior to the coronal suture through a scalp incision. The injection rate was 2 μL/minute, and sixty seconds after the completion of the injection, the needle was withdrawn and the incision sutured. Approximately 25 days following intracranial tumor implantation, each animal was imaged using a 7 Tesla small animal MRI system (Bruker BioSpin, Ettlinger, Germany)(FIG. 11). For contrast enhancement, T1-weighted images were obtained following Gd-DTPA administration via tail vein injection (0.05-2.5 mmol/kg) over a period of 5-7 seconds. This analysis indicated that human GBM tumors could be generated in rat brain.

To facilitate monitoring, GBM cells, which express Renilla luciferase (viral particles purchased from GenTarget, San Diego, Calif.) are used so that intracranial tumor formation is non-invasively measured by bioluminescence. Renilla luciferase (RLuc) catalyzes coelenterazine, which is distinct from the luciferin substrate used to image pHSP70-induction of F-Luc reporter. Due to the use of different substrates, these distinct luciferases are evaluated using two separate processes (F-Luc for pHSP70 activation in stem cells and R-Luc for tumor cell growth). Rats bearing invasive orthotopic GBM tumors are injected with stem cells transformed to express sTRAIL ˜20 days after tumor implantation and imaging confirmation of tumor growth (bioluminescence and/or MRI). The anti-tumor effects of the stem cells secreting sTRAIL are determined by injecting various amounts of recombinant stem cells (1-10×10⁶) at various locations relative to the implanted tumor. For example, recombinant stem cells that have been enriched via FACS to express sTRAIL or controls (transduced with reporter construct) are directly injected into the tumor using the same injection site and coordinates as the tumor implantation. This demonstrates anti-tumor efficacy and the minimal number of cells needed at the tumor site to see a therapeutic effect. In addition, separate groups of rats are injected with recombinant stem cells (or controls) 3 mm from the tumor site, contralateral to the tumor site, and systemically. Forty-eight hours after stem cell implantation, target sites are heated to approximately 43° C. using HIFU. To accomplish stringently controlled HIFU heating, a MRI/MR thermometry-guided HIFU system (RK100, FUS Instruments Inc., Toronto, Calif.) is used to deliver high power ultrasound energy to the rat brain for pHSP70 induction. The system can deliver ultrasound exposures ranging from high-power continuous sonications (thermal coagulation) to pulsed sonications for applications such as transcranial therapy, drug delivery and activation. The system probe is a spherically focused ultrasound transducer with a center frequency of 1 MHz and a focal spot size around 1-2 mm in diameter and 5-6 mm in length. During treatment, the focused spot is placed against the rat right superior cranium (site of the tumor) on the bed of the Siemens Skyra 3T scanner. The acoustic intensity around the focused spot and the HIFU-induced hyperthermia are controlled and monitored in real-time by using MR-thermometry. The MR thermometry allows real-time temperature mapping with a spatial resolution of 1.88×1.882×5 mm³ every 5 seconds. Real-time temperature mapping non-invasively acquired by MR-thermometry is further calibrated by a MR compatible fiber optical thermometer.

pHSP70 activation is confirmed via bioluminescent imaging and sTRAIL expression is confirmed using immunohistochemistry. For bioluminescent imaging, rats are imaged post-heating on the IVIS bioluminescent scanner (PerkinElmer) immediately after i.v. injection of 150 mg/kg D-luciferin, the F-Luc substrate. ROI are drawn over the heated and unheated tumors and quantified. In addition to bioluminescence, subgroups of rats are sacrificed at fixed time points after HIFU treatments (0, 12, 24, and 48 hours) and the injected stem cells are localized relative to tumors using multicolor fluorescence microscopy, as the stem cells constitutively express RFP in addition to their pHSP70-induced GFP. Induction is measured by calculating the ratio of induced GFP expressing cells compared to RFP expressing stem cells, which is constitutively expressed even by non-activated stem cells. It is expected that a temperature of 43° C. can be precisely controlled in normal brain tissue and tumors in rats.

After confirming therapeutic expression by activated stem cells, the effects of the stem cells that express sTRAIL on tumor growth (compared to control that will be transduced with reporter only or not heated) are evaluated. The antitumor effects are monitored for up to 200 days after injection by measuring tumor volume by MRI (weekly), bioluminescence (weekly), and survival (Kaplan-Meier analysis). In addition, the animals are monitored 3 times/week after therapy to ensure that there are no unexpected clinical consequences caused by stem cell injection or HIFU treatments. It is expected that only the stem cells expressing sTRAIL under HSP70 promoter control will demonstrate anti-tumor activity, in contrast to stem cells infected with reporter genes or unheated stem cells.

### Example 6

### Thermal Control of Cytokine Production and Migration of Recombinant Stem Cells

Due to tumor-tropic migratory properties, stem cells can serve as vehicles for the delivery of effective, targeted treatment to isolated tumors and to metastatic disease. For example, human mesenchymal stem cells have been transformed to deliver biologic anti-glioma agents to gliomas, including interferon β, S-TRAIL, and oncolytic viruses, with demonstrable survival advantages. The therapeutic efficacy of stem cell therapy for a tumor relies on the number of stem cells that travel from the site of delivery to reach the tumor area. Moreover, stem cell migration is highly affected by cytokine secretion. For example, the migration of mesenchymal stem cells is dependent upon the different cytokine/receptor pairs SDF-1/CXCR4, SCF/c-Kit, HGF/c-Met, VEGF/VEGFR, PDGF/PDGFR, MCP-1/CCR2, and HMGB1/RAGE. Stromal cell-derived factor 1 (SDF-1) and its receptor CXC chemokine receptor-4 (CXCR4) are important mediators of neuron stem cell recruitment to tumors. Engineering strategies can be used to control cytokine expression in tumor tissue to attract the stem cell migration. The ability to enhance stem cell delivery to tumor tissues would significantly reduce the number of cells required to achieve a therapeutic effect, and presumably provide better outcomes for patients. In this respect, the present composition and method can spatially and temporally control the induction of specific cytokine production in stem cells that have accumulated at the target site. The cytokine produced will be designed to attract more recombinant stem cells to the target site leading to amplification of the effect.

To demonstrate control of cytokine production, NSCs and MSCs were transduced with pLenti-HSP70 (F-Luc-2A-cytokines)-RSV (RFP-BSD)(FIG. 12A) and screened for blasticidin resistance. Blasticidin-resistant NSCs and MSCs permanently demonstrated red fluorescence (FIG. 12B). The recombinant cells encoding HSP70-driven cytokines were heated by heat block, ultrasound or infrared light to 43° C. for 20 minutes to induce expression of cytokines (TNFα, VEGF or TGFβ1) (FIG. 13), Recombinant cells were subsequently seed into wells of the lower compartment of a TRANSWELL chamber, and NSCs and MSCs expressing GFP were seeded into the upper compartment. Cells migrating into the lower compartment were counted under a fluorescence microscope. As demonstrated in FIG. 14, heat-induced hMSCs (i.e., cells expressing cytokines) significantly attracted the stem cell migration from the upper TRANSWELL compartment to the plate as compared to the control hMSCs without induction. Similarly, heat-induced NSCs (i.e., cells expressing cytokines) significantly attracted NSCs migration from the upper TRANSWELL compartment to the plate as compared to the control NSCs without induction (FIG. 15).

### Example 7

### Use of Cytokines to Attract a Second Amplified Wave of Therapeutic Stem Cells

Tumor-tropic migration of stem cells is mediated by tumor-secreted soluble factors (A. Belmadani et al., J. Neurosci. 26(12):3182-3191 (2006)). Therefore, recombinant stem cells, which secrete these pro-migratory factors, can induce the migration of a second wave of recombinant stem cells to tumors. Accordingly, a first wave of stem cells are produced to selectively express a stem cell attracting chemokine/cytokine under the control of HIFU. Once this first wave reaches the tumor, HIFU is used to temporally induce stem cells to express the pro-migratory soluble factor in and around the tumor. This induced soluble factor consequently attracts a second wave of therapeutically engineered stem cells significantly more effectively to the tumor vicinity than would the tumor alone, hence amplifying the therapeutic potential of the second wave of stem cells. By way of illustration, TNFα is used as the cytokine to attract stem cell migration, because (i) TNFα is a well-known inflammatory factor and has been shown to effectively attract stem cells (G. Kitange et al., J. Neurooncol. 92(1):23-31 (2009); J. Sarkaria et al., Mol. Cancer Ther. 6(3):1167-1174 (2007); J. Sarkaria et al., Clin. Cancer Res. 12(7 Pt 1):2264-2271 (2006)), (ii) TNFα has the potential to open BBB (N. Tsao et al., J Med. Microbial. 50(9):812-821 (2001); R. Reyes et al., J Neurosurg. 110(6):1218-1226 (2009); J. Mullin et al., Cancer Res. 50(7):2172-2176 (1990); M. Lopez-Ramirez et al., J. Immunol. 189(6):3130-3139 (2012)), and (iii) the pHSP70-controlled construct effectively increases NSC migration in vitro without killing NSCs (FIG. 15). Thus, a first wave of stem cells encoding TNFα can attract an amplified second wave of stem cell migration toward the tumor. In addition to TNFα, it is posited that other inflammatory factors, e.g., SDF-1α (K. Carbajal et al., Proc. Natl. Acad. Sci. USA 107(24):11068-11073 (2010); J. Imitola et al., Proc. Natl. Acad. Sci. USA 101(52):18117-18122 (2004)); tumor-associated growth factors, e.g., scatter factor/hepatocyte growth factor (SF/HGF), TGFα, and fibroblast growth factor (FGF)(O. Reese, et al., Neuro-Oncol. 7(4):476-484 (2005)); and endothelial cell-derived chemo-attractants such as PDGF-BB, RANTES, I-TAC, NAP-2, GROα, Ang-2, and M-CSF (N. Schmidt et al., Brain Res. 1268:24-37 (2009)), can be used to attract the second wave of therapeutic stem cells in viva.

The effect of cytokines to amplify the migratory capacity of a second wave of recombinant stem cells was demonstrated in vitro using conditioned media from cells infected with a virus that expresses TNFα. A lentivirus encoding TNFα under the control of the HSP70 promoter was produced. This vector, designated pLenti-pHSP70:TNFα/FLuc-pRSV:RFP (see FIG. 12A), was transduced into SF767 human glial tumor cells. The recombinant SF767 cells were mildly heated at 43° C. for 30 minutes to induced TNFα expression and shown to secrete TNFα into the medium (FIG. 16A). Furthermore, it was demonstrated that conditioned media prepared from these heat-activated cells infected with pLenti-pHSP70:TNFα/F-Luc-pRSV:RFP vector significantly increased directional NSC migration compared to the controlled conditioned medium from cells without heat induction (FIG. 16B).

### Example 8

### Use of HIFU-Induced Production of Cytokines to Focally Permeabilize the BBB to Additional Systemic Therapies

One impediment to the treatment of GBM is the poor penetration of therapeutics through tumor-BBB. It has been shown that cytokines and chemokines can have profound effects on tumor-associated BBB penetration and can enable utilization of efficacious anti-cancer therapies that are currently not used to treat GBM due to exclusion by the BBB (N. Tsao et al., J. Med. Microbial. 50(9):812-821 (2001); R. Reyes et al., J Neurosurg. 110(6):1218-1226 (2009); J. Mullin et al., Cancer Res. 50(7):2172-2176 (1990); M. Lopez-Ramirez et al., J Immunol. 189(6)1130-3139 (2012)). Thus, HIFU-activated pro-inflammatory factor production by recombinant stem cells in the GBM tumor region can be used to significantly increase BBB permeability and tumor concentration of systemically administered drugs. Importantly, strictly controlled HIFU induction to control cytokine expression from recombinant stem cells enables this approach because of the short-lived and controllable induction, making it much less likely to cause unintended adverse effects or promoting tumor growth.

A lentiviral expression plasmid, pLenti-Hsp70 (F-Luc-2A-TNFα)-RSV (RFP-BSD) (FIG. 17A), which contains the heat-inducible HSP70 promoter driving expression of firefly luciferase (F-Luc) and tumor necrosis factor alpha (TNFα), and RSV promoter driving expression of red fluorescent protein (RFP) and blasticidin selection marker (BSD), was constructed. Mesenchymal stem cells (MSCs) were engineered by transduction with this plasmid construct by lentiviral vector (GenTarget, San Diego, Calif.). Heat-activated gene expression of TNFα was confirmed and optimized in terms of temperature and duration of time using a water bath in vitro. For in vivo study, MSCs transduced with HSP70 (F-Luc-2A-TNFα)-RSV (RFP-BSD) were stereotactically implanted into the brains of athymic nude rats (1×10⁶ cells per rat). 2 days after cell implantation, the area of injection site was heated to 43° C. by HIFU under guidance of MRI for half an hour to induce TNFα expression. The luciferase expression was monitored by bioluminescence after injection of luciferin. After 48 hours, opening of the BBB was confirmed on T1-weighted image after intravenous injection of the MRI contrast agent (Magnevist, 0.125 mmol/kg) by tail veil. Rats implanted with MSCs-HSP70 (Luc-2A-TNFα) without HIFU treatment and rats implanted with MSCs-HSP70 (Luc-2A-GFP) with HIFU treatment were used as the controls.

MSCs were successfully transduced with pLenti-HSP70 (F-Luc-2A-TNFα)-RSV (RFP-BSD), and screened by blasticidin. The engineered MSCs cells permanently demonstrated red fluorescence (FIG. 17B). HSP70-driven transgene expression was tightly dependent on the temperature and duration of time. Activation at 43° C. for 15 minutes led to highest expression of TNFα and F-Luc (FIG. 17C). The engineered MSCs were then stereotactically implanted into the rat brain followed by MRI-guided HIFU activation. As shown in FIG. 18A, rats implanted with HSP70 (Luc-2A-GFP) or MSCs-HSP70 (Luc-2A-TNFα) with HIFU treatment at 43° C. for 20 minutes demonstrated significantly stronger bioluminescence signal in the brain compared to rats implanted with MSCs-HSP70 (Luc-2A-TNFα) without HIFU treatment. Quantification of the region of interest revealed 10 times higher F-Luc expression in the brain of rat after HIFU activation. Following the bioluminescence imaging, MR contrast agent was injected through the tail vein to monitor changes in BBB permeability in contrast-enhanced T1-weighed images. Significant MRI signal enhancement was observed in the targeted regions of the brain in rats implanted with MSCs-HSP70 (Luc-2A-TNFα) with HIFU treatment compared to the controls (FIG. 18B). Quantification of the region of interest demonstrated 3 times higher MRI signal intensity indicating increased BBB permeability to the MRI contrast agent in the brain of rat implanted with MSCs-HSP70 (Luc-2A-TNFα) with HIFU treatment (P<0.01) compared to the controls (FIG. 19).

The foregoing is illustrative of the present invention, and is not to be construed as limiting thereof. The invention is defined by the following claims, with equivalents of the claims to be included therein.

1. A recombinant nucleic acid, said recombinant nucleic acid comprising:
(a) a heat-inducible promoter operatively associated with
(b) a nucleic acid encoding an active agent, wherein said active agent is (1) a stem-cell attracting chemokine, or (ii) a blood-brain barrier opening protein or peptide.

2. The recombinant nucleic acid of claim 1, wherein said promoter is a heat inducible protein promoter.

3. The recombinant nucleic acid of claim 2, wherein said heat inducible promoter is selected from the group consisting of an HSP70 promoter, an HSP90 promoter, an HSP60 promoter, an HSP27 promoter, an HSP25 promoter, a ubiquitin promoter, a growth arrest gene promoter, and a DNA Damage gene promoter.

4. The recombinant nucleic acid of claim 1, wherein said active agent is a stem-cell attracting chemokine selected from the group consisting of TNF-alpha, stromal cell-derived factor 1 alpha, tumor-associated growth factors, transforming growth factor alpha, fibroblast growth factor, endothelial cell-derived chemoattractants, vascular endothelial growth factor (VEGF), and stem cell factor (SCF); subject to the proviso that VEGF is excluded when said recombinant nucleic acid is not in a stem cell transformed therewith.

5. The recombinant nucleic acid of claim 1, wherein said active agent is a blood-brain barrier opening protein or peptide selected from the group consisting of bradykinin, thrombin, endothelin-1, substance P, platelet activating factor, cytokines, macrophage inflammatory proteins, and complement-derived polypeptide C3a-desArg.

6. A vector containing a recombinant nucleic acid of claim 1.

7. The vector of claim 6, wherein said vector is a viral or retroviral vector.

8. A stem cell transformed with a heterologous recombinant nucleic acid of claim 1.

9. The stem cell of claim 8, wherein said stem cell is an embryonic stem cell, adult stem cell, or induced pluripotent stem cell.

10. A composition comprising a stem cell of claim 8 in a pharmaceutically acceptable carrier

11. A method of preparing a tissue for therapeutic treatment in a subject in need thereof, comprising:
(a) parenterally administering to the subject preconditioning stem cells that migrate to said tissue, said stem cells containing a recombinant nucleic acid, said recombinant nucleic acid comprising a nucleic acid encoding a stem-cell attracting chemokine operably associated with a heat-inducible promoter; and then
(b) selectively heating said tissue sufficient to induce the expression of said stem-cell attracting chemokine therein in an amount effective to enhance the migration of therapeutic stem cells subsequently administered parenterally to said subject.

12. The method of claim 11, wherein said tissue is brain, breast, skin, prostate, lung, retina, muscle, liver, pancreatic, skeletal, or cartilage tissue.

13. The method of claim 11, wherein said tissue is a neoplastic tissue.

14. The method of claim 13, wherein said neoplastic tissue is brain tumor, breast cancer, skin cancer, prostate cancer or lung cancer tissue.

15. The method of claim 11, wherein said stem cells are embryonic stem cells, adult stem cells, or induced pluripotent stem cells.

16. The method of claim 11, wherein said promoter is a heat inducible protein promoter.

17. The method of claim 16, wherein said heat inducible promoter is selected from the group consisting of an HSP70 promoter, an HSP90 promoter, an HSP60 promoter, an HSP27 promoter, an HSP25 promoter, a ubiquitin promoter, a growth arrest gene promoter, and a DNA Damage gene promoter.

18. The method of claim 11, wherein said stem-cell attracting chemokine selected from the group consisting of TNF-alpha, stromal cell-derived factor 1alpha, tumor-associated growth factors, transforming growth factor alpha, fibroblast growth factor, endothelial cell-derived chemoattractants, vascular endothelial growth factor (VEGF), and stem cell factor (SCF).

19. The method of claim 11, wherein said selectively heating step is carried out by ultrasound, laser, radiofrequency, microwave or water bath.

20. The method of claim 19, wherein said selectively heating step is carried out by high intensity focused ultrasound.

21. The method of claim 11, wherein said parenterally administering is a systemic administering step.

22. A method of treating a tissue in a subject in need thereof, comprising:
(a) parenterally administering to a subject therapeutic stem cells that migrate to said tissue, said stem cell containing a recombinant nucleic acid, said recombinant nucleic acid comprising a nucleic acid encoding a therapeutic agent operably associated with a heat-inducible promoter; and then
(b) selectively heating said tissue sufficient to induce the expression of said therapeutic agent therein in a treatment-effective amount.

23. The method of claim 22, wherein said therapeutic agent is selected from the group consisting of a toxin, a fragment of a toxin, a drug-metabolizing enzyme, and an inducer of apoptosis.

24. The method of claim 22, wherein the therapeutic agent is (a) a toxin is selected from the group consisting of a bacterial toxin, a plant toxin, a fungal toxin and a combination thereof; (b) a drug-metabolizing enzyme comprising kinase; or (c) an inducer of apoptosis selected from the group consisting of PUMA; BAX; BAK; BcI-XS; BAD; BIM; BIK; BID; HRK; Ad E1B; an ICE-CED3 protease; TRAIL; SARP-2; and apoptin.

25. The method of claim 22, wherein said tissue is brain, breast, skin, prostate, lung, retina, muscle, liver, pancreatic, skeletal, or cartilage tissue.

26. The method of claim 22, wherein said tissue is a neoplastic tissue.

27. The method of claim 26, wherein said neoplastic tissue is brain tumor, breast cancer, skin cancer, prostate cancer or lung cancer tissue.

28. The method of claim 22, wherein said stem cells are embryonic stem cells, adult stem cells, or induced pluripotent stem cells.

29. The method of claim 22, wherein said promoter is a heat inducible protein promoter.

30. The method of claim 29, wherein said heat inducible promoter is selected from the group consisting of an HSP70 promoter, an HSP90 promoter, an HSP60 promoter, an HSP27 promoter, an HSP25 promoter, a ubiquitin promoter, a growth arrest gene promoter, and a DNA Damage gene promoter.

31. The method of claim 22, wherein said selectively heating step is carried out by ultrasound, laser, radiofrequency, microwave or water bath.

32. The method of claim 19, wherein said selectively heating step is carried out by high intensity focused ultrasound.

33. The method of claim 22, wherein said parenterally administering is a systemic administering step.

34. A method of preparing for treatment and treating a tissue in a subject in need thereof, comprising:
(a) parenterally administering to the subject preconditioning stem cells that migrate to said tissue, said stem cells containing a recombinant nucleic acid, said recombinant nucleic acid comprising a nucleic acid encoding a stem-cell attracting chemokine operably associated with a heat-inducible promoter; then
(b) selectively heating said tissue sufficient to induce the expression of said stem-cell attracting chemokine therein in an amount effective to enhance the migration of therapeutic stem cells subsequently parenterally administered to said subject; then
(c) parenterally administering to a subject therapeutic stem cells that migrate to said tissue, said stem cells optionally containing a recombinant nucleic acid, said recombinant nucleic acid comprising a nucleic acid encoding a therapeutic agent operably associated with a heat-inducible promoter; and then optionally:
(d) selectively heating said tissue sufficient to induce the expression of said therapeutic agent therein in a treatment-effective amount.

35. The method of claim 34, wherein said stem-cell attracting chemokine is selected from the group consisting of TNF-alpha, stromal cell-derived factor 1 alpha, tumor-associated growth factors, transforming growth factor alpha, fibroblast growth factor, endothelial cell-derived chemoattractants, vascular endothelial growth factor (VEGF), and stem cell factor (SCF).

36. The method of claim 34, wherein said therapeutic agent is selected from the group consisting of a toxin, a fragment of a toxin, a drug-metabolizing enzyme, and an inducer of apoptosis.

37. The method of claim 34, wherein the therapeutic agent is (a) a toxin is selected from the group consisting of a bacterial toxin, a plant toxin, a fungal toxin and a combination thereof; (b) a drug-metabolizing enzyme comprising kinase; or (c) an inducer of apoptosis selected from the group consisting of PUMA; BAX; BAK; BcI-XS; BAD; BIM; BIK; BID; HRK; Ad E1B; an ICE-CED3 protease; TRAIL; SARP-2; and apoptin.

38. The method of claim 34, wherein said tissue is brain, breast, skin, prostate, lung, retina, muscle, liver, pancreatic, skeletal, or cartilage tissue.

39. The method of claim 34, wherein said tissue is a neoplastic tissue.

40. The method of claim 39, wherein said neoplastic tissue is brain tumor, breast cancer, skin cancer, prostate cancer or lung cancer tissue.

41. The method of claim 34, wherein either or both said stem cells are embryonic stem cells, adult stem cells, or induced pluripotent stem cells.

42. The method of claim 34, wherein said promoter is a heat inducible protein promoter.

43. The method of claim 42, wherein either or both said heat inducible promoter is selected from the group consisting of an HSP70 promoter, an HSP90 promoter, an HSP60 promoter, an HSP27 promoter, an HSP25 promoter, a ubiquitin promoter, a growth arrest gene promoter, and a DNA Damage gene promoter.

44. The method of claim 34, wherein either or both said selectively heating step is carried out by ultrasound, laser, radiofrequency, microwave or water bath.

45. The method of claim 44, wherein either or both said selectively heating step is carried out by high intensity focused ultrasound.

46. The method of claim 34, wherein either or both said parenterally administering is a systemic administering step.

47. A method of increasing blood-brain barrier permeability of selected brain tissue in a subject in need thereof, comprising:
(a) parenterally administering to the subject stem cells that migrate to the brain tissue, said stem cells containing a recombinant nucleic acid, said recombinant nucleic acid comprising a nucleic acid encoding a barrier-opening protein or peptide operably associated with a heat-inducible promoter; and then
(b) selectively heating said selected brain tissue sufficient to induce the expression of said barrier-opening protein or peptide in an amount effective to increase the permeability of the blood-brain barrier in said selected brain tissue.

48. The method of claim 47, wherein said selected tissue is neoplastic tissue.

49. The method of claim 47, wherein said stem-cell attracting chemokine selected from the group consisting of TNF-alpha, stromal cell-derived factor 1 alpha, tumor-associated growth factors, transforming growth factor alpha, fibroblast growth factor, endothelial cell-derived chemoattractants, vascular endothelial growth factor (VEGF), and stem cell factor (SCF).

50. The method of claim 47, wherein said blood-brain barrier opening protein or peptide is selected from the group consisting of bradykinin, thrombin, endothelin-1, substance P, platelet activating factor, cytokines, macrophage inflammatory proteins, and complement-derived polypeptide C3a-desArg.

51. The method of claim 47, wherein said stem cells are embryonic stem cells, adult stem cells, or induced pluripotent stem cells.

52. The method of claim 47, wherein said promoter is a heat inducible protein promoter.

53. The method of claim 52, wherein said heat inducible promoter is selected from the group consisting of an HSP70 promoter, an HSP90 promoter, an HSP60 promoter, an HSP27 promoter, an HSP25 promoter, a ubiquitin promoter, a growth arrest gene promoter, and a DNA Damage gene promoter.

54. A method of claim 47, wherein said stem cells are administered in an amount effective to increase the cytotoxic effect of a therapeutic agent in said subject, said method further comprising administering the therapeutic agent to the subject.

55. A method of claim 54, wherein said therapeutic agent is selected from the group consisting of temozolomide (“Tmz”), VP-16, paclitaxel, carboplatin, tumor necrosis factor-related apoptosis-inducing ligand (“TRAIL”), troglitazone (“TGZ”), pioglitazone (“PGZ”), rosiglitazone (“RGZ”), and ciglitazone (“CGZ”), procarbazine, vincristine, BCNU, CCNU, thalidomide, irinotecan, isotretinoin, imatinib, etoposide, cisplatin, daunorubicin, doxorubicin, methotrexate, mercaptopurine, fluorouracil, hydroxyurea, vinblastine, and combinations thereof.

56. (canceled)
